(19) United States
(12) Patent Application Publication
(10) Pub. No.: US 2003/0158254 A1
(43) Pub. Date: Aug. 21, 2003

(54) Engineering absorption of therapeutic compounds via colonic transporters

(75) Inventors: Noa Zerangue, San Mateo, CA (US); Kenneth C. Cundy, Redwood City, CA (US); Mark A. Gallop, Los Altos, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: XenoPort, Inc., Santa Clara, CA

(21) Appl. No.: 10/351,291

(22) Filed: Jan. 23, 2003

Related U.S. Application Data
(60) Provisional application No. 60/351,808, filed on Jan. 24, 2002.

(51) Int. Cl. A61K 31/325; A61K 38/17; C07K 14/47

(52) U.S. Cl. 514/484; 514/12

(57) ABSTRACT

Methods of modifying therapeutic compounds such as drugs to be substrates for active transporters expressed in epithelial cells lining the lumen of the human colon are disclosed. The transporters expressed in the human colon include the sodium dependent multi-vitamin transporter (SMVT), and monocarboxylate transporters 1 and 4 (MCT 1 and MCT 4). The modified compounds can themselves be pharmacologically active, or upon cleavage of a chemical moiety after uptake from the colon, can be metabolized to form a compound that is pharmacologically active (e.g., a prodrug). The modified compounds disclosed herein are suitable for use in extended release oral dosage forms, particularly those that release drug over periods of greater than about 2-4 hours following administration.

### CROSS-REFERENCE TO RELATED APPLICATION

Priority of U.S. Patent Application Serial No. 60/351,808, filed Jan. 24, 2002 is claimed and the disclosures of the application are incorporated by reference in its entirety for all purposes.

### BACKGROUND

It is often desirable to extend the effect of an administered dose of medicinal compounds. This may be done for convenience and improved rate of compliance, as for example when a drug with short circulating half life may be administered once rather than several times per day. It may also be done to improve the efficacy or lower the toxicity of a drug by buffering the rapid rise and fall of blood levels produced by the frequent administration of a short-lived compound—thereby producing a more tonic profile of blood concentration. The period of time that a compound administered orally is maintained at efficacious blood and tissue concentration is determined by several factors: the intrinsic half life of the compound in the circulation (and the target tissue), which depends on the kinetics of metabolism, excretion and distribution; the regimen of administration, and the kinetics of absorption. One strategy to extend the residence time of a compound administered as a single oral dose is to delay the absorption of the compound in the intestine. A means of accomplishing this is by slow release formulation, such as slowly dissolving tablets, bioerodable encapsulation, or an osmotic controlled release oral dosage form such as those sold by ALZA Corporation under the trademark OROS®. However, sustained release compositions are effective to achieve sustained release following oral administration only for certain types of agents.

### SUMMARY OF THE CLAIMED INVENTION

The invention provides a pharmaceutical composition comprising an agent linked to a conjugate moiety to form a conjugate, formulated with a pharmaceutical carrier for sustained or delayed release of the conjugate, wherein the conjugate has a higher Vmax for a transporter expressed in plasma membranes of epithelial cells lining a human colon than the agent alone.

Optionally, the Vmax of the conjugate is at least two-fold or ten-fold higher than that of the agent alone. Optionally, the agent substantially lacks capacity to be taken up as a substrate for a transporter expressed in plasma membranes of epithelial cells lining a human colon.

Optionally, the pharmaceutical carrier comprises a polymeric material, such as a polymeric material degraded by a change in pH, exposure to an enzyme or a change in pressure. Optionally, the polymeric material is a non-degradable osmotic membrane. Optionally, the agent is linked by a cleavable linkage to the conjugate moiety to form the conjugate.

Optionally, the conjugate is not a substrate for a transporter expressed in plasma membranes of epithelial cells lining a human small intestine. Optionally, the conjugate is substantially incapable of passive transport through the human intestine. Optionally, the conjugate has a greater Vmax for a transporter expressed in plasma membranes of epithelial cells lining a human small intestine than the agent alone. Optionally, the agent is further linked to a second conjugate moiety to form a modified conjugate, and the modified conjugate has a reduced Vmax for a transporter expressed in plasma membranes of epithelial cells lining a human small human intestine than the conjugate alone. Optionally, the agent is further linked to a second conjugate moiety to form a modified conjugate, and the modified conjugate has a reduced capacity for passive transport through a human intestine than the conjugate alone. Optionally, the agent is further linked to a second conjugate moiety to form a modified conjugate, and the modified conjugate has an increased Vmax for a transporter expressed in plasma membranes of epithelial cells lining a human small human intestine than the conjugate alone.

Optionally, the transporter is selected from the group consisting of solute carrier transporters, facilitative diffusion transporters, active transporters, and pumps. Optionally, the agent is selected from gabapentin, pregabalin and pharmaceutically acceptable salts thereof. Optionally, the conjugate is gabapentin pivaloxymethyl carbamate, gabapentin phenylacetoxymethyl carbamate or gabapentin benzoyloxymethyl carbamate. Optionally, the agent is selected from L-dopa, carbidopa and a pharmaceutically acceptable salts thereof. Optionally, the transporter is a transporter described in Tables 1 or 2. Optionally, the transporter is any of ATBO, CAT-1, FATP4, MCT1, MCT4, NADC1, NADC2, OCTN2, PEPT1, PGT, RFC, SAT-1, SAT-6, SMVT, SUT2 and SVCT1. Optionally, the transporter effects transport through an apical plasma membrane or a basolateral plasma membrane of epithelial cells lining the colon, or both. Optionally, the transporter affects transport through an apical plasma membrane of epithelial cells lining the colon.

The invention further provides a pharmaceutical composition comprising a therapeutic agent linked to a conjugate moiety to form a conjugate, formulated with a pharmaceutical carrier in an oral dosage form which upon oral administration to a human releases at least a portion of the conjugate within the colon of the human, wherein the conjugate has a higher Vmax for a transporter selected from MCT1, MCT4 and SMVT than the agent alone.

The invention further provides a method of formulating an agent. The method involves linking the agent to a conjugate moiety to form a conjugate, wherein the conjugate moiety has a greater Vmax for a transporter expressed in plasma membranes of epithelial cells lining a human colon than the agent alone; and formulating the conjugate with a pharmaceutical carrier as a sustained or delayed release pharmaceutical composition.

Optionally, the Vmax of the conjugate is at least two-fold or ten-fold higher than that of the agent alone. Optionally, the agent substantially lacks capacity to be taken up as a substrate for a transporter expressed in plasma membranes of epithelial cells lining a human colon.

Optionally, the pharmaceutical carrier comprises a polymeric material, such as one degraded by a change in pH, exposure to an enzyme or a change in pressure. Optionally, the polymeric material is a non-degradable osmotic membrane. Optionally, the agent is linked by a cleavable linkage to the conjugate moiety to form a conjugate.

Optionally, the conjugate is not a substrate for a transporter expressed in plasma membranes of epithelial cells lining a human small intestine. Optionally, the conjugate is substantially incapable of passive transport through the human intestine. Optionally, the conjugate has a greater Vmax for a transporter expressed in plasma membranes of epithelial cells lining a small intestine than the agent alone.

Optionally, the agent is further linked to a second conjugate moiety to form a modified conjugate, and the modified conjugate has a reduced Vmax for a transporter expressed in plasma membranes of epithelial cells lining a small human intestine than the conjugate alone. Optionally, the agent is further linked to a second conjugate moiety to form a modified conjugate, and the modified conjugate has a reduced capacity for passive transport through a human intestine than the conjugate alone. Optionally, the agent is further linked to a second conjugate moiety to form a modified conjugate, and the modified conjugate has an increased Vmax for a transporter expressed in plasma membranes of epithelial cells lining a small human intestine than the conjugate alone.

Optionally, the transporter is selected from the group consisting of solute carrier transporters, facilitative diffusion transporters, active transporters, and pumps. Optionally, the agent is selected from gabapentin, pregabalin and pharmaceutically acceptable salts thereof. Optionally, the agent is selected from L-dopa, carbidopa and pharmaceutically acceptable salts thereof. Optionally, the transporter is a transporter described in Table 1 or 2. Optionally, the transporter is selected from the group consisting of ATBO, CAT-1, FATP4, MCT1, MCT4, NADC1, NADC2, OCTN2, PEPT1, PGT, RFC, SAT-1, SAT-6, SMVT, SUT2 and SVCT1. Optionally, the transporter effects transport through an apical plasma membrane or a basolateral plasma membrane of epithelial cells lining the colon, or both. Optionally, the transporter effects transport through apical plasma membranes of epithelial cells lining a human colon.

The invention further provides a method of delivering an agent. Such a method involves orally administering to a patient a pharmaceutical composition comprising an agent linked to a conjugate moiety to form a conjugate, formulated with a pharmaceutical carrier for sustained or delayed release of the agent or conjugate, wherein the conjugate has a higher Vmax for a transporter expressed in plasma membranes of epithelial cells lining a human colon than the agent alone, whereby the conjugate is released from the carrier in the colon of the patient, and passes through the transporter into the circulation.

Optionally, the Vmax of the conjugate is at least two-fold or ten-fold higher than that of the agent alone. Optionally, the agent substantially lacks capacity to be taken up as a substrate by a transporter expressed in plasma membranes of epithelial cells lining a human colon.

Optionally, the pharmaceutical carrier comprises a polymeric material. Optionally, the polymeric material is degraded by a change in pH, exposure to an enzyme or a change in pressure. Optionally, the polymeric material is a non-degradable osmotic membrane.

Optionally, the agent is linked by a cleavable linkage to the conjugate moiety to form the conjugate. Optionally, the conjugate is not a substrate for a transporter expressed in plasma membranes of epithelial cells lining a human small intestine. Optionally, the conjugate is substantially incapable of passive transport through the human intestine. Optionally, the conjugate has a greater Vmax for a transporter expressed in plasma membranes of epithelial cells lining a human small intestine than the agent alone.

Optionally, the agent is further linked to a second conjugate moiety to form a modified conjugate, and the modified conjugate has a reduced Vmax for a transporter expressed in plasma membranes of epithelial cells lining a human small intestine than the conjugate alone. Optionally, the agent is further linked to a second conjugate moiety to form a modified conjugate, and the modified conjugate has a reduced capacity for passive transport through a human intestine than the conjugate alone. Optionally, the agent is further linked to a second conjugate moiety to form a modified conjugate, and the modified conjugate has an increased Vmax for a transporter expressed in plasma membranes of epithelial cells lining a human small intestine than the conjugate alone.

Optionally, the transporter is selected from the group consisting of solute carrier transporters, facilitative diffusion transporters, active transporters, and pumps. Optionally, the agent is selected from gabapentin, pregabalin and pharmaceutically acceptable salts thereof. Optionally, the conjugate is gabapentin pivaloxymethyl carbamate, gabapentin phenylacetoxymethyl carbamate or gabapentin benzoyloxymethyl carbamate. Optionally, the agent is selected from L-dopa, carbidopa and pharmaceutically acceptable salts thereof. Optionally, the transporter is a transporter described in Table 1. Optionally, the transporter is selected from the group consisting of ATBO, CAT-1, FATP4, MCT1, MCT4, NADC1, NADC2, OCTN2, PEPT1, PGT, RFC, SAT-1, SAT-6, SMVT, SUT2 and SVCT1.

The invention further provides a method of screening agents, conjugates or conjugate moieties for oral delivery. The method involves providing a cell expressing a transporter expressed in the human colon, the transporter being situated in the plasma membrane of the cell; contacting the cell with an agent, conjugate or conjugate moiety; and determining whether the agent, conjugate or conjugate moiety passes through the plasma membrane via the transporter. Optionally, the agent or conjugate is substantially incapable of passive diffusion through the plasma membrane.

The invention further provides a method of delivering an agent. The method involves orally administering to a patient a pharmaceutical composition comprising an agent, optionally, linked to a conjugate moiety to form a conjugate, formulated with a pharmaceutical carrier for sustained or delayed release of the agent or conjugate, wherein the agent, conjugate moiety (if present) or conjugate (if present) has been screened to determine that it is a substrate for a transporter expressed in plasma membranes of epithelial cells lining a human colon.

Optionally, the screening can be performed by providing a cell expressing a transporter expressed in plasma membranes of epithelial cells lining a human colon, the transporter being situated in the plasma membrane of the provided cell; contacting the provided cell with an agent, conjugate or conjugate moiety; and determining whether the agent, conjugate or conjugate moiety passes through the membrane via the transporter.

Optionally, the pharmaceutical carrier comprises a polymeric material, such as one degraded by a change in pH, exposure to an enzyme or a change in pressure. Optionally, the polymeric material is a non-degradable osmotic membrane.

Optionally, the agent or conjugate (if present) is not a substrate for a transporter expressed in plasma membranes of epithelial cells lining a human small intestine. Optionally, the agent or conjugate (if present) is substantially incapable of passive transport through the human intestine. Optionally, the transporter is selected from the group consisting of solute carrier transporters, facilitative diffusion transporters, active transporters, and pumps. Optionally, the transporter is a transporter described in Table 1 or 2. Optionally, the transporter is selected from the group consisting of ATBO, CAT-1, FATP4, MCT1, MCT4, NADC1, NADC2, OCTN2, PEPT1, PGT, RFC, SAT-1, SAT-6, SMVT, SUT2 and SVCT1. Optionally, the transporter effects transport through an apical plasma membrane or a basolateral plasma membrane of epithelia cells lining the colon, or both. Optionally, the transporter effects transport through apical plasma membranes of epithelial cells lining the colon.

### BRIEF DESCRIPTION OF THE FIGURES

FIG. 1 shows uptake of Compound I by HEK cells in the presence and absence of a transporter inhibitor phloretin.

FIG. 2 compares transport of gabapentin conjugate Compound V in the presence and absence of PEPT1/PEPT2 inhibitor Lys(ε-Dansyl)-Leu.

FIG. 3A compares colonic uptake of Compounds I, II and III. Uptake is determined from plasma concentration of gabapentin. FIG. 3B shows pharmacokinetic parameters.

FIG. 4 compares uptake into the plasma of Compound V following oral and intracolonic administration.

FIG. 5 shows examples of natural drugs that are substrates for polyamine transporters.

### DEFINITIONS

A “transporter protein” is a protein that has a direct or indirect role in transporting a molecule into and/or through a cell. This term includes solute carrier transporters, co transporters, counter transporters, uniporters, symporters, antiporters, pumps, equilibrative transporters, concentrative transporters; and other proteins mediating active transport, energy-dependent transport, facilitated diffusion, exchange mechanisms, specific absorption mechanisms. The term includes, for example, membrane-bound proteins that recognize a substrate and affect its entry into, or exit from a cell by a carrier-mediated transporter or by receptor-mediated transport. These proteins are sometimes referred to as transporter proteins. The term also includes intracellularly expressed proteins that participate in trafficking of substrates through or out of a cell. The term also includes proteins or glycoproteins exposed on the surface of a cell that do not directly transport a substrate but bind to the substrate holding it in proximity to a receptor or transporter protein that effects entry of the substrate into or through the cell. Examples of carrier proteins include: the intestinal and liver bile acid transporters, dipeptide transporters, oligopeptide transporters, simple sugar transporters (e.g., SGLT1), phosphate transporters, monocarboxylic acid transporters, P-glycoprotein transporters, organic anion transporters (OAT), and organic cation transporters. Examples of receptor-mediated transport proteins include: viral receptors, immunoglobulin receptors, bacterial toxin receptors, plant lectin receptors, bacterial adhesion receptors, vitamin transporters and cytokine growth factor receptors.

Absorption by passive diffusion refers to uptake of an agent that is not mediated by a specific transporter protein. An agent that is substantially incapable of passive diffusion has a permeabilty across a standard cell monolayer (e.g., Caco-2) in vitro of less than 5×10⁻⁶ cm/sec, and usually less than 1×10⁻⁶ cm/sec in the absence of an efflux mechanism.

A “substrate” of a transport protein is a compound whose uptake into or passage through a cell is facilitated at least in part by a transporter protein.

The term “ligand” of a transport protein includes substrates and other compounds that bind to the transport protein without being taken up or transported through a cell. Some ligands by binding to the transport protein inhibit or antagonize uptake of the substrate or passage of substrate through a cell by the transport protein. Some ligands by binding to the transport protein promote or agonize uptake or passage of the compound by the transport protein or another transport protein. For example, binding of a ligand to one transport protein can promote uptake of a substrate by a second transport protein in proximity with the first transport protein.

The term “agent” is used to describe a compound that has or may have a pharmacological activity. Agents include compounds that are known drugs, compounds for which pharmacological activity has been identified but which are undergoing further therapeutic evaluation, and compounds that are members of collections and libraries that are to be screened for a pharmacological activity.

An agent is “orally active” if it can exert a pharmaceutical activity when administered via an oral route.

A “conjugate” refers to a compound comprising an agent and a chemical moiety bound thereto, which moiety by itself or in combination with the agent renders the conjugate a substrate for active transport. The chemical moiety may or may not be subject to cleavage from the agent upon uptake and metabolism of the conjugate in the patient's body. In other words, the moiety may be cleavably bound to the agent or non-cleavably bound to the agent. The bond can be a direct (i.e., covalent) bond or the bond can be through a linker. In cases where the bond/linker is cleavable by metabolic processes, the agent, or a further metobolite of the agent, is the therapeutic entity. In cases where the bond/linker is not cleavable by metabolic processes, the conjugate is the therapeutic entity. Most typically, the conjugate comprises a prodrug having a metabolically cleavable moiety, where the conjugate itself does not have pharmacological activity but the agent to which the moiety is cleavably bound does have pharmacological activity. Typically, the moiety facilitates therapeutic use of the agent by promoting uptake of the conjugate via a transporter. Thus, for example, a conjugate comprising an agent and a conjugate moiety may have a Vmax for a transporter that is at least 2, 5, 10, 20, 50 or 100-fold higher than that of the agent alone. A conjugate moiety can itself be a substrate for a transporter or can become a substrate when linked to the agent (e.g., valacyclovir, an L-valine ester prodrug of the antiviral drug acyclovir). Thus, a conjugate formed from an agent and a moiety can have higher uptake activity than either the agent or the moiety alone.

A “pharmacological” activity means that an agent exhibits an activity in a screening system that indicates that the agent is or may be useful in the prophylaxis or treatment of a disease. The screening system can be in vitro, cellular, animal or human. Agents can be described as having pharmacological activity notwithstanding that further testing may be required to establish actual prophylactic or therapeutic utility in treatment of a disease.

Vmax and Km of a compound for a transporter are defined in accordance with convention. Vmax is the number of molecules of compound transported per second at saturating concentration of the compound. Km is the concentration of the compound at which the compound is transported at half of Vmax. In general, a high value of Vmax is desirable for a substrate of a transporter. A low value of Km is desirable for transport of low concentrations of a compound, and a high value of Km is desirable for transport of high concentrations of a compound. Vmax is affected both by the intrinsic turnover rate of a transporter (molecules/transporter protein) and transporter density in plasma membrane which depends on expression level. For these reasons, the intrinsic capacity of a compound to be transported by a particular transporter is usually expressed as the ratio Vmax of the compound/Vmax of a control compound known to be a substrate for the transporter.

“Sustained release” refers to release of a therapeutic or prophylactic amount of the drug or an active metabolite thereof into the systemic blood circulation over a prolonged period of time relative to that achieved by oral administration of a conventional formulation of the drug. “Delayed release” refers to release of a therapeutic or prophylactic amount of the drug or an active metabolite thereof into the systemic blood circulation at a later period of time relative to that achieved by oral administration of a conventional formulation of the drug.

A transporter is expressed in a particular tissue, e.g., the colon, when expression can be detected by by mRNA analysis, protein analysis, antibody histochemistry, or functional transport assays. Typically, detectable mRNA expression is at a level of at least 0.01% of the of beta actin in the same tissue or at least 0.2% of glyceraldehyde-3-phosphate dehydrogenase (GAPDH) mRNA. Preferred transporters exhibit levels of expression in the desired tissue (e.g., colon) of at least 0.1, or 1 or 10% of that of GAPDH or beta actin. Of these two metrics, GAPDH is preferred as it is more consistent than beta actin. Conversely a transporter is not expressed in a particular tissue (e.g., the small intestine) if expression is not detectable above experimental error by any of the above techniques. Thus, transporters that are not expressed in particular tissue exhibit express levels less than 0.1% of GAPDH or beta actin, and usually less than 0.01% of GAPDH or beta actin.

The phrases “specifically binds” when referring to a protein or “specifically immunoreactive with” when referring to an antibody, refers to a binding reaction which is determinative of the presence of the protein in the presence of a heterogeneous population of proteins and other biologics. Thus, under designated conditions, a specified ligand binds preferentially to a particular protein and does not bind in a significant amount to other proteins present in the sample. A molecule such as antibody that specifically binds to a protein often has an association constant of at least 10⁵ M⁻¹, 10⁶ M⁻¹ or 10⁷ M⁻¹, preferably 10⁸ M⁻¹ to 10⁹ M⁻¹, and more preferably, about 10¹⁰ M⁻¹ to 10¹¹ M⁻¹ or higher. However, some substrates of transporters, PEPT1 and MCT's in particular, have much lower affinities of the order of 10-10³ M⁻¹ and yet the binding can still be shown to be specific. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

Another example of algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra.). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. For identifying whether a nucleic acid or polypeptide is within the scope of the invention, the default parameters of the BLAST programs are suitable. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix. The TBLATN program (using protein sequence for nucleotide sequence) uses as defaults a word length (W) of 3, an expectation (E) of 10, and a BLOSUM 62 scoring matrix. (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

### DETAILED DESCRIPTION

Disclosed herein are methods and pharmaceutical compositions for sustained delivery of agents via one or more transporters expressed in the human colon. The methods and pharmaceutical compositions disclosed herein take advantage of a number of transporter proteins expressed in the human colon. Methods of sustained-release oral delivery are effective only if the administered agent remains for an extended period in a portion of the intestine capable of absorbing the compound. Such absorption across the gut wall can be via either “passive” diffusion, by active transport mechanisms such as solute carrier transporters and/or by endocytosis, or by combinations of passive and active transport. For those agents absorbed primarily by non-specific passive diffusion, any segment of the intestine is effective to absorb the compound. Thus, the agent can be continuously absorbed at different places in the small intestine and colon as it is released. Many therapeutic compounds however exhibit poor or no passive diffusion across the gut wall, with the result that oral bioavailability of such compounds is insufficient for effective therapy. Other therapeutic compounds are transported primarily by one or more transporters expressed in the small intestine and not in the colon. These agents are thus taken up only for the relatively short period in which a sustained release composition resides in the small intestine, and any agent that is released downstream from the small intestine (i.e., in the colon) is not absorbed and is excreted. Disclosed herein are methods to design, select or modify agents such that they are substrates for a transporter expressed in the human colon. Such agents or their modified forms can thus be taken up during the relatively long period during which a sustained release composition passes through the human colon.

I. Transporters Expressed in the Human Colon

The human small intestine is a convoluted tube about twenty feet in length that runs between the stomach and large intestine. The small intestine is subdivided into the duodenum, the jejunum and the ileum. The large intestine is about 5 feet in length and runs from the ileum to the anus. The large intestine is divided into the caecum, colon and the rectum. The colon is itself divided into four parts, the ascending, transverse, descending and the sigmoid flexure. In general, on orally ingested agent spends about 1-6 hr in the stomach, about 2-4 hr in the small intestine, and about 8 to 18 hr in the colon. Thus, the greatest period of time for sustained release of an agent occurs when the agent is passing through the colon.

Some transporters expressed in the human colon are not expressed in other human tissues. Some transporters expressed in the human colon are also expressed in the human small intestine (e.g., organic anion transporters). Some transporters expressed in the human colon are also expressed in human tissues other than the small intestine (e.g., polyamine transporter). Some transporters are expressed in the apical plasma membrane of epithelial cells and some transporters in the basolateral membrane of these epithelial cells, and some transporters are expressed in both.

Transporters expressed in the apical plasma membrane are preferred. Table 1 shows transporters expressed in the apical membrane of epithelial cells lining the human colon. Table 2 shows transporters expressed in the human colon for which it has not yet been determined whether they are expressed in the apical or basolateral membrane. Tables 1 and 2 also indicate whether the transporters are expressed in the colon of species other than humans. Transporters expressed in additional species are preferred. In both Tables 1 and 2, expression means that mRNA of a transporter is expressed at least at the 0.2% of glyceraldehyde-3-phosphate dehydrogenase mRNA.

Preferred transporters include ATBO, CAT-1, FATP4, MCT1, MCT4 (Monocarboxylate transporters), NADC1, NADC2, OCTN2, PEPT1, PGT, RFC, SAT-1, SAT-6, SMVT (sodium dependent multi-vitamin transporter), SUT2 and SVCT 1. Particularly preferred transporters are MCT1, MCT4, ATBO, OCTN2, NADC1 and NADC2. In some methods, the transporter is a transporter expressed in the colon other than SMVT.

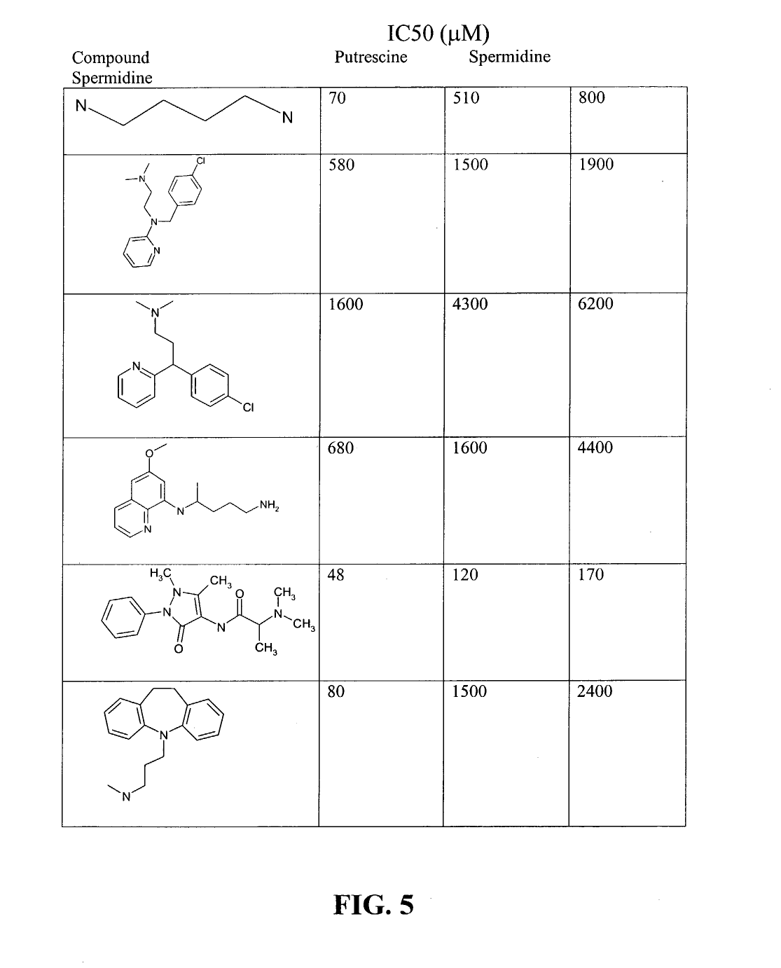

Some examples of natural drugs that are substrates for polyamine transporters are shown in FIG. 5. Some transporters expressed in the human colon are expressed in the human small intestine and in at least one other human tissue (e.g., PEPT1).

GenBank accession numbers for the transporters are given in the table above. Unless otherwise apparent from the context, reference to a transporter includes the amino acid sequence described in or encoded by the GenBank reference, and, allelic, cognate and induced variants and fragments thereof retaining essentially the same transporter activity. Usually such variants show at least 90% sequence identity to the exemplary Genbank nucleic, acid or amino acid sequence.

II. Strategies for Sustained Release

Agents having pharmacological activity are designed, selected or modified to be substrates for at least one transporter expressed in the colon. In some instances, an agent as a result of chemical design or selection from a pool of candidate agents, can inherently be a substrate for such a transporter. In other instances, an agent that substantially lacks substrate activity for a transporter (i.e., no detectable activity) is modified to become a substrate by addition of a conjugate moiety. The modified agent is referred to as a conjugate. If the conjugate moiety of a conjugate can be detached from the agent after administration to release the agent, then the conjugate can be referred to as a prodrug. In some instances, the substrate activity of an agent or conjugate is specific to a transporter expressed only in the colon, and the agent or conjugate is substantially incapable of passive diffusion. In other instances, the agent or conjugate is a substrate for one or more colon transporters and also is a substrate for a transporter expressed in the small intestine, and/or is capable of passive diffusion. In still other instances, the agent or conjugate is a substrate for a colon transporter, and a small intestine transporter and a transporter expressed in a target issue.

The choice of transporter depends in part on the structure of the conjugate to be administered. Typically, the targeted transporter is one having natural substrates with structural similarities to the conjugate to be administered. The choice of transporter also depends on the dosage of agent, since agents which require higher blood concentrations to be therapeutically effective will require targeting transporters with greater uptake capacity. In general, a transporter exhibiting a lower K_{M} (i.e., a higher affinity) for the conjugate is generally desirable.

The choice of transporter also depends on the desired pharmacokinetics. If the agent or conjugate is a substrate for a transporter expressed in the colon but not a substrate for passive diffusion or for a transporter expressed in the small intestine, then no absorption of the agent or conjugate occurs until it has passed through the stomach and small intestine into the colon. The rate of uptake in the colon can be further controlled by selecting a transporter with appropriate Vmax. The lower the Vmax the slower the agent or conjugate is absorbed in the colon. Conversely, if the agent or conjugate is a substrate for passive diffusion or a transporter that is expressed in the small intestine, then absorption occurs both in the small intestine and the colon. The agent or conjugate can also be designed or selected to be, or not be, a substrate for a transporter expressed in tissues other than the small intestine. Such can be advantageous in situations in which targeting of the agent or conjugate to a particular tissue is either desired or to be avoided.

In some instances, the desired specificity of an agent or conjugate can be achieved simply by selecting and screening for substrate capacity to a single transporter. For example, if one wants an agent or conjugate to be a be a substrate for a transporter expressed in the colon and a transporter expressed in the small intestine, then one can select a transporter expressed in both. In other instances, however, two modifications of an agent are necessary to confer the desired substrate specificity. For example, an agent can be linked to one conjugate moiety to render the agent a substrate for one transporter, and to a second conjugate moiety to render the agent a substrate for a second transporter. Alternatively, an agent can be linked to one conjugate moiety to render the agent a substrate for one transporter, and to a second conjugate moiety to prevent the agent from being a substrate for a second transporter or for passive diffusion. For example, linkage to a polar conjugate moiety can render an agent incapable of passive diffusion.

The agent or conjugate can be formulated with an appropriate pharmaceutical carrier as a sustained release composition to ensure gradual release of the agent or conjugate as it passes through the small intestine and colon. Alternatively, the agent or conjugate can be formulated with a pharmaceutical carrier as a delayed release composition. Such a composition releases relatively little, if any, agent or conjugate in the initial period of administration during which the agent or conjugate passes through the stomach and small intestine. After a period of time sufficient to allow passage through the stomach and small intestine, the agent is then released from the delayed release composition. The release can occur rapidly or slowly as the delayed release composition passes through the colon. For some substrate specificities and consequent pharmacokinetic profiles, sustained release formulation is not necessary. For example, if an agent or conjugate is specific for a transporter expressed only in the colon and is incapable of passive diffusion, then essentially all of the agent or conjugate reaches the colon substantially irrespective of whether it is formulated as a sustained-release composition. Particularly, if the colon transporter selected has a relatively low Vmax, uptake of the agent or conjugate occurs throughout the length of the colon.

All of the above strategies lead to delivery of a substantial proportion of the agent or conjugate to the colon where the agent or conjugate is available for uptake by a colon transporter. The substantial proportion is preferably at least 25%, 50% or 75% of the total agent or conjugate administered. The proportion can be measured by comparing the concentration of an agent or conjugate in blood over time following oral administration compared with administration directly to the colon. A device for administering a drug directly to the colon is described by U.S. Pat. No. 4,904,474. The proportion can also be estimated by plotting blood concentration versus time following oral uptake and comparing the area under the curve before and after six hours after administration. The area under the curve before six hours is an approximation of uptake in the stomach and small intestine and that after six hours is an estimate of uptake in the colon. The area under the curve after six hours is preferably at least 25%, 50% or 75% of the total area under the curve. Alternatively, compositions can be evaluated by exposing the compositions to artificial gastric and/or artificial small intestinal fluid in vitro and determining how much agent or conjugate is retained in the composition after a certain period. The composition of these fluids is provided by The United States Pharmacopoeia, (Twentieth Revision, 1980) at p 1105. Preferably, at least 25%, 50% or 75% of agent or conjugate is retained after exposure to 4 hours of artificial gastric fluid and 2 hours of small intestinal fluid.

Using a sustained release oral dosage, the conjugate or agent is preferably released from the dosage form over a period of at least about 6 hours, more preferably, over a period of at least about 8 hours, and most preferably, over a period of at least about 12 hours. Further, the dosage form preferably releases from 0 to 20% of the conjugate in 0 to 2 hours, from 20 to 50% of the conjugate in 2 to 12 hours, from 50 to 85% of the conjugate in 3 to 20 hours and greater than 75% of the conjugate in 5 to 18 hours. Further, the sustained release oral dosage form further provides a concentration of the conjugate in the blood plasma of the patient over time, which curve has an area under the curve (AUC) that is, ideally, proportional to the dose of the conjugate administered, and a maximum concentration Cₘₐₓ. The Cₘₐₓ is less than 75%, and is preferably, less than 60%, of the Cₘₐₓ obtained from administering an equivalent dose of the conjugate from an immediate release oral dosage form, and the AUC is substantially the same as the AUC obtained from administering an equivalent dose of the conjugate from an immediate release oral dosage form. Preferably, the time period in which an effective therapeutic concentration of drug is maintained in the blood is increased by at least 25%, 50% or 75% relative to the period for an immediate release formulation. Preferably, the time period during which drug is absorbed into the blood is increased by at least 25%, 50% or 75% relative to an immediate release formulation. For a delayed release oral dosage form, the dosage form preferably releases at least 50, or 75% of the composition after a period of at least 2-6 hours from administration. For example, release of 75% of the composition between 6 and 10 hours after administration is suitable. The time at which Cₘₐₓ occurs is preferably delayed by 2-6 hr relative to the time of the Cₘₐₓ obtained from administering an equivalent dose of the conjugate or agent from an immediate release oral dosage form. The AUC is substantially the same as the AUC obtained from administering an equivalent dose of the conjugate or agent from an immediate release oral dosage form. The magnitude of Cmax may be the same, higher of lower than the Cmax obtained from administering an equivalent dose of the conjugate or agent from an immediate release oral dosage form.

III. Methods of Identifying Agents or Conjugate Moieties that are Substrates of a Transporter

Agents known or suspected to have pharmacological activity can be screened directly for their capacity to act as substrates of one or more of the colon expressed transporters described above. Alternatively, conjugate moieties can be screened as substrates, and the conjugate moieties linked to agents having known or suspected pharmacological activity. In such methods, the conjugate moieties can be linked to an agent or other molecule during the screening process. If another molecule is used, the molecule is sometimes chosen to resemble the structure of an agent ultimately intended to be linked to the conjugate moiety for pharmaceutical use. The screening can be performed either in vitro using cells expressing the transporter or in vivo by direct delivery of an agent or conjugate to the colon.

In some methods, the cells are transfected with DNA encoding a transporter. Oocytes and CHO cells, for example, are suitable for transfection. In other methods, natural cells expressing a transporter are used. Human embryonic kidney cells (HEKs), and CaCo-2 cells express many transporter proteins that are also expressed in the human colon. In some methods, the cells only express a colon-expressed transporter. In other methods, cells express a transporter of the invention in combination with other transporters. In still other methods, agents, conjugate moieties or conjugates are screened on different cells expressing different transporters. Agents, conjugate moieties or conjugates can be screened either for specificity for one transporter or for capacity to be substrates to several transporters. Agents, conjugate moieties or conjugates with specificity for a particular transporter can be useful for limiting uptake to certain tissues or avoiding interaction between drugs. Agents, conjugate moieties or conjugates that are substrates for multiple transporters are useful for maximum uptake.

Internalization of a compound evidencing passage through transporters can be detected by detecting a signal from within a cell from any of a variety of reporters. The reporter can be as simple as a label such as a fluorophore, a chromophore, a radioisotope, Confocal imaging can also be used to detect internalization of a label as it provides sufficient spatial resolution to distinguish between fluorescence on a cell surface and fluorescence within a cell; alternatively, confocal imaging can be used to track the movement of compounds over time. In another approach, internalization of a compound is detected using a reporter that is a substrate for an enzyme expressed within a cell. Once the complex is internalized, the substrate is metabolized by the enzyme and generates an optical signal or radioactive decay that is indicative of uptake. Light emission can be monitored by commercial PMT-based instruments or by CCD-based imaging systems. In addition, assay methods utilizing LC/MS detection of the transported compounds or electrophysiological signals indicative of transport activity are also employed. Agents and conjugates can also be screened in vivo by administration of the agent or conjugate directly into the colon of an animal and monitoring passage of the agent or conjugate into the blood.

In some methods, multiple agents, conjugate moieties or conjugate moieties are screened simultaneously and the identity of each agent, conjugate or conjugate moiety is tracked using tags linked to the agents or conjugate moieties. In some methods, a preliminary step is performed to determine binding of an agent, conjugate or conjugate moiety to a transporter. Although not all agents, conjugates or conjugate moieties that bind to a transporter are substrates of the transporter, observation of binding is an indication that allows one to reduce the number of candidate substrates from an initial repertoire. In some methods, the transport rate of an agent, conjugate or conjugate moiety is tested in comparison with the transport rate of a reference substrate for that transporter. The comparison can either be performed in separate parallel assays in which an agent, conjugate or conjugate moiety under test and the reference substrate are compared for uptake on separate samples of the same cells. Alternatively, the comparison can be performed in a competition format in which an agent, conjugate or conjugate moiety under test and the reference substrate are applied to the same cells. Typically, the agent, conjugate or conjugate moiety and the reference substrate are differentially labeled in such assays.

In such comparative assays, the Vmax of an agent, conjugate or conjugate moiety, tested can be compared with that of the reference substrate. If an agent, conjugate moiety or conjugate has a Vmax of at least 1%, 5%, 10%, 20%, and most preferably at least 50% of the reference substrate for the transporter then the agent, conjugate moiety or conjugate can be considered to be a substrate for the transporter. In general, the higher the Vmax of the agent, conjugate moiety or conjugate relative to that of the reference substrate the better. Therefore, agents, conjugate moieties or conjugates having Vmax's of at least 50%, 100%, 150% or 200% (i.e., two-fold) of the Vmax of the reference substrate for the transporter are screened in some methods. The agents to which conjugate moieties are linked can by themselves show little or no detectable substrate activity for the transporter (e.g., Vmax relative to that of a reference substrate of less than 0.1% or 1%).

In some methods, the Vmax of an agent, conjugate moiety or conjugate is also determined relative to the reference substrate for a second transporter. Such screening may reveal that the agent, conjugate moiety or conjugate is a better substrate for one transporter than another. The relative capacities of a substrate for two transporters can be compared by a comparison of the ratios of Vmax of the agent, conjugate moiety or conjugate for the respective transporters.

IV. Agents, Conjugates and Conjugate Moieties to be Screened

Compounds constituting agents, conjugates or conjugate moieties to be screened can be naturally occurring or synthetic molecules. Natural sources include sources such as, e.g., marine microorganisms, algae, plants, and fungi. Alternatively, compounds to be screened can be from combinatorial libraries of agents, including peptides or small molecules, or from existing repertories of chemical compounds synthesized in industry, e.g., by the chemical, pharmaceutical, environmental, agricultural, marine, cosmeceutical, drug, and biotechnological industries. Compounds can include, e.g., pharmaceuticals, therapeutics, environmental, agricultural, or industrial agents, pollutants, cosmeceuticals, drugs, heterocyclic and other organic compounds, lipids, glucocorticoids, antibiotics, peptides, sugars, carbohydrates, and chimeric molecules.

Some compounds to be screened are variants of known transporter substrates. Some compounds to be screened are bile salts or acids, steroids, ecosanoids, or natural toxins or analogs thereof, as described by Smith, Am. J. Physiol. 2230, 974-978 (1987); Smith, Am. J. Physiol. 252, G479-G484 (1993); Boyer, Proc. Natl. Acad. Sci. USA 90, 435-438 (1993); Fricker, Biochem. J. 299, 665-670 (1994); Ficker, Biochem J. 299, 665-670 (1994); Ballatori, Am. J. Physiol. 278

V. Linkage of Agents to Conjugate Moieties

Conjugates of this invention can be prepared by either by direct conjugation of an agent to a conjugate moiety, wherein the resulting covalent bond is cleavable in vivo, or by covalently coupling a difunctionalized linker precursor with an agent to a conjugate moiety. The linker precursor is selected to contain at least one reactive functionality that is complementary to at least one reactive functionality on the agent and at least one reactive functionality on the conjugate moiety. Such complementary reactive groups are well known in the art as illustrated below:

COMPLEMENTARY BINDING CHEMISTRIES

| First Reactive Group | Second Reactive Group | Linkage |
|---|---|---|
| hydroxyl | carboxylic acid | ester |
| hydroxyl | haloformate | carbonate |
| thiol | carboxylic acid | thioester |
| thiol | haloformate | thiocarbonate |
| amine | carboxylic acid | amide |
| hydroxyl | isocyanate | carbamate |
| amine | haloformate | carbamate |
| amine | isocyanate | urea |
| carboxylic acid | carboxylic acid | anhydride |
| hydroxyl | phosphorus acid | phosphonate or |
| | | phosphate ester |

In addition to the complementary chemistry of the functional groups on the linker to both the agent and conjugate moiety, the linker (when employed) is also selected to be cleavable in vivo. Cleavable linkers are well known in the art and are selected such that at least one of the covalent bonds of the linker that attaches the agent to the conjugate moiety can be broken in vivo thereby providing for the agent or active metabolite thereof to be available to the systemic blood circulation. The linker is selected such that the reactions required to break the cleavable covalent bond are favored at the physiological site in vivo which permits agent (or active metabolite thereof) release into the systemic blood circulation.

The selection of suitable cleavable linkers to provide effective concentrations of the agent or active metabolite thereof for release into the systemic blood circulation can be evaluated using endogenous enzymes in standard in vitro assays to provide a correlation to in vivo cleavage of the agent or active metabolite thereof from the conjugate, as is well known in the art. It is recognized that the exact cleavage mechanism employed is not critical to the methods of this invention provided, of course, that the conjugate cleaves in vivo in some form to provide for the agent or active metabolite thereof for sustained release into the systemic blood circulation.

In another approach, a conjugate moiety and agent are each attached to moieties having mutual affinity for each other (e.g., avidin or streptavidin and biotin, or hexahistidine and Ni²⁺). In another approach, both agent and conjugate moiety are linked to a solid or particulate support. Examples of such supports include nanoparticles (see, e.g., U.S. Pat. Nos. 5,578,325 and 5,543,158), molecular scaffolds, liposomes (see, e.g., Deshmuck, D. S., et al., Life Sci. 28:239-242 (1990), and Aramaki, Y., et al., Pharm. Res. 10:1228-1231 (1993), protein cochleates (stable protein-phospholipid-calcium precipitates; see, e.g., Chen et al., J. Contr. Rel. 42:263-272 (1996), and clathrate complexes. These supports can be used to attach other active molecules. Certain supports such as nanoparticles can also be used to encapsulate desired compounds. An agent can be linked to a support via a cleavable linkage allowing separation of the agent after uptake through a transporter.

Examples of cleavable linkers suitable for use as described above include nucleic acids with one or more restriction sites, or peptides with protease cleavage sites (see, e.g., U.S. Pat. No. 5,382,513). Other exemplary linkers that can be used are also described in International Patent Application WO 02/44324; European Patent Application 188,256; U.S. Pat. Nos. 4,671,958; 4,659,839; 4,414,148; 4,669,784; 4,680,338, 4,569,789 and 4,589,071 each of which is incorporated in its entirety for all purposes.

There are many existing drugs for which uptake can be improved through the colon. Drugs suitable for conversion to prodrugs that are capable of uptake from the colon typically contain one or more of the following functional groups to which a promoiety may be conjugated: primary or secondary amino groups, hydroxyl groups, carboxylic acid groups, phosphonic acid groups, or phosphoric acid groups.

Examples of drugs containing carboxyl groups include, for instance, angiotensin-converting enzyme inhibitors such as alecapril, captopril, 1-[4-carboxy-2-methyl-2R,4R-pentanoyl]-2,3-dihydro-2S-indole-2-carboxylic acid, enalaprilic acid, lisinopril, N-cyclopentyl-N-[3-[(2,2-dimethyl-1-oxopropyl)thio]-2-methyl-1-oxopropyl]glycine, pivopril, quinaprilat, (2R, 4R)-2-hydroxyphenyl)-3-(3-mercaptopropionyl)-4-thiazolidinecarboxylic acid, (S) benzamido-4-oxo-6-phenylhexenoyl-2-carboxypyrrolidine, [2S-1[R*(R*))]] 2α, 3αβ, 7αβ]-1 [2-[[1-carboxy-3-phenylpropyl]-amino]-1-oxopropyl]octahydro-1H-indole-2-carboxylic acid, [3S-1[R*(R*))]], 3R*]-2-[2-[[1-carboxy-3-phenylpropyl]-amino]-1-oxopropyl]-1,2,3,4-tetrahydro-3-isoquinolone carboxylic acid, and tiopronin; cephalosporin antibiotics such as cefaclor, cefadroxil, cefamandole, cefatrizine, cefazedone, cefazuflur, cefazolin, cefbuperazone, cefixime, cefinenoxime, cefmetazole, cefodizime, cefonicid, cefoperazone, ceforanide, cefotaxime, cefotefan, cefotiam, cefoxitin, cefpimizole, cefpirome, cefpodoxime, cefroxadine, cefsulodin, cefpiramide, ceftazidime, ceftezole, ceftizoxime, ceftriaxone, cefuroxime, cephacetrile, cephalexin, cephaloglycin, cephaloridine, cephalosporin, cephanone, cephradine, and latamoxef; penicillins such as amoxycillin, ampicillin, apalcillin, azidocillin, azlocillin, benzylpencillin, carbenicillin, carfecillin, carindacillin, cloxacillin, cyclacillin, dicloxacillin, epicillin, flucloxacillin, hetacillin, methicillin, mezlocillin, nafcillin, oxacillin, phenethicillin, piperazillin, sulbenicllin, temocillin, and ticarcillin; thrombin inhibitors such as argatroban, melagatran, and napsagatran; influenza neuraminidase inhibitors such as zanamivir and BCX-1812; non-steroidal antiinflammatory agents such as acametacin, alclofenac, alminoprofen, aspirin (acetylsalicylic acid), 4-biphenylacetic acid, bucloxic acid, carprofen, cinchofen, cinmetacin, clometacin, clonixin, diclenofac, diflunisal, etodolac, fenbufen, fenclofenac, fenclosic acid, fenoprofen, ferobufen, flufenamic acid, flufenisal, flurbiprofin, fluprofen, flutiazin, ibufenac, ibuprofen, indomethacin, indoprofen, ketoprofen, ketorolac, lonazolac, loxoprofen, meclofenamic acid, mefenamic acid, 2-(8-methyl-10,11-dihydro-11-oxodibenz[b,f]oxepin-2-yl)propionic acid, naproxen, nifluminic acid, O-(carbamoylphenoxy)acetic acid, oxoprozin, pirprofen, prodolic acid, salicylic acid, salicylsalicylic acid, sulindac, suprofen, tiaprofenic acid, tolfenamic acid, tolmetin and zopemirac; prostaglandins such as ciprostene, 16-deoxy-16-hydroxy-16-vinyl prostaglandin E₂, 6,16-dimethylprostaglandin E₂, epoprostostenol, meteneprost, nileprost, prostacyclin, prostaglandins E₁, E₂, or F_{2α}, and thromboxane A₂; quinolone antibiotics such as acrosoxacin, cinoxacin, ciprofloxacin, enoxacin, flumequine, naladixic acid, norfloxacin, ofloxacin, oxolinic acid, pefloxacin, pipemidic acid, and piromidic acid; other antibiotics such as aztreonam, imipenem, meropenem, and related carbopenem antibiotics.

Representative drugs containing amine groups include: acebutalol, albuterol, alprenolol, atenolol, bunolol, bupropion, butopamine, butoxamine, carbuterol, cartelolol, colterol, deterenol, dexpropanolol, diacetolol, dobutamine, exaprolol, exprenolol, fenoterol, fenyripol, labotolol, levobunolol, metolol, metaproterenol, metoprolol, nadolol, pamatolol, penbutalol, pindolol, pirbuterol, practolol, prenalterol, primidolol, prizidilol, procaterol, propanolol, quinterenol, rimiterol, ritodrine, solotol, soterenol, sulfiniolol, sulfinterol, sulictidil, tazaolol, terbutaline, timolol, tiprenolol, tipridil, tolamolol, thiabendazole, albendazole, albutoin, alendronate, alinidine, alizapride, amiloride, aminorex, aprinocid, cambendazole, cimetidine, cisapride, clonidine, cyclobenzadole, delavirdine, efegatrin, etintidine, fenbendazole, fenmetazole, flubendazole, fludorex, gabapentin, icadronate, lobendazole, mebendazole, metazoline, metoclopramide, methylphenidate, mexiletine, neridronate, nocodazole, oxfendazole, oxibendazole, oxmetidine, pamidronate, parbendazole, pramipexole, prazosin, pregabalin, procainamide, ranitidine, tetrahydrazoline, tiamenidine, tinazoline, tiotidine, tocainide, tolazoline, tramazoline, xylometazoline, dimethoxyphenethylamine, N-[3(R)-[2-piperidin-4-yl)ethyl]-2-piperidone-1-yl]acetyl-3(R)-methyl-β-alanine, adrenolone, aletamine, amidephrine, amphetamine, aspartame, bamethan, betahistine, carbidopa, clorprenaline, chlortermine, dopamine, L-Dopa, ephrinephrine etryptamine, fenfluramine, methyldopamine, norepinephrine, tocainide, enviroxime, nifedipine, nimodipine, triamterene, norfloxacin, and similar compounds such as pipedemic acid, 1-ethyl-6-fluoro-1,4dihydro-4-oxo-7-(1-piperazinyl)-1,8-napthyridine-3-carboxylic acid, 1-cyclopropyl-6-fluoro-1, and 4-dihydro-4-oxo-7-(piperazinyl)-3-quinolinecarboxylic acid.

Representative drugs containing hydroxy groups include: steroidal hormones such as allylestrenol, cingestol, dehydroepiandrosteron, dienostrol, diethylstilbestrol, dimethisteron, ethyneron, ethynodiol, estradiol, estron, ethinyl estradiol, ethisteron, lynestrenol, mestranol, methyl testosterone, norethindron, norgestrel, norvinsteron, oxogeston, quinestrol, testosterone, and tigestol; tranquilizers such as dofexazepam, hydroxyzin, lorazepam, and oxazepam; neuroleptics such as acetophenazine, carphenazine, fluphenazine, perphenyzine, and piperaetazine; cytostatics such as aclarubicin, cytarabine, decitabine, daunorubicin, dihydro-5-azacytidine, doxorubicin, epirubicin, estramustin, etoposide, fludarabine, gemcitabine, 7-hydroxychlorpromazin, nelarabine, neplanocin A, pentostatin, podophyllotoxin, tezacitabine, troxacitabine, vinblastin, vincristin, and vindesin; hormones and hormone antagonists such as buserilin, gonadoliberin, icatibrant, and leuprorelin acetate; antihistamines such as terphenadine; analgesics such as diflunisal, naproxol, paracetamol, salicylamide, and salicyclic acid; antibiotics such as azidamphenicol, azithromycin, camptothecin, cefamandol, chloramphenicol, clarithromycin, clavulanic acid, clindamycin, demeclocyclin, doxycyclin, erythromycin, gentamycin, imipenem, latamoxef, metronidazole, neomycin, novobiocin, oleandomycin, oxytetracyclin, tetracycline, thiamenicol, and tobramycin; antivirals such as acyclovir, d4C, ddC, DMDC, Fd4C, FddC, FMAU, FTC, 2′-fluoro-ara-dideoxyinosine, ganciclovir, lamivudine, penciclovir, SddC, stavudine, 5-trifluoromethyl-2′-deoxyuridine, zalcitabine, and zidovudine; bisphosphonates such as EB-1053, etidronate, ibandronate, olpadronate, residronate, YH-529, and zolendronate; protease inhibitors such as ciprokiren, enalkiren, ritonavir, saquinavir, and terlakiren; prostaglandins such as arbaprostil, carboprost, misoprostil, and prostacydin; antidepressives such as 8-hydroxychlorimipramine and 2-hydroxyimipramine; antihypertonics such as sotarol and fenoldopam; anticholinerogenics such as biperidine, procyclidin and trihexyphenidal; antiallergenics such as cromolyn; glucocorticoids such as betamethasone, budenosid, chlorprednison, clobetasol, clobetasone, corticosteron, cortisone, cortodexon, dexamethason, flucortolon, fludrocortisone, flumethasone, flunisolid, fluprednisolon, flurandrenolide, flurandrenolon acetonide, hydrocortisone, meprednisone, methylpresnisolon, paramethasone, prednisolon, prednisol, triamcinolon, and triamcinolon acetonide; narcotic agonists and antagonists such as apomorphine, buprenorphine, butorphanol, codein, cyclazocin, hydromorphon, ketobemidon, levallorphan, levorphanol, metazocin, morphine, nalbuphin, nalmefen, naloxon, nalorphine, naltrexon, oxycodon, oxymorphon, and pentazocin; stimulants such asmazindol and pseudoephidrine; anaesthetics such as hydroxydion and propofol; β-receptor blockers such as acebutolol, albuterol, alprenolol, atenolol, betazolol, bucindolol, cartelolol, celiprolol, cetamolol, labetalol, levobunelol, metoprolol, metipranolol, nadolol, oxyprenolol, pindolol, propanolol, and timolol; α-sympathomimetics such as adrenalin, metaraminol, midodrin, norfenefrin, octapamine, oxedrin, oxilofrin, oximetazolin, and phenylefrin; β-sympathomimetics such as bamethan, clenbuterol, fenoterol, hexoprenalin, isoprenalin, isoxsuprin, orciprenalin, reproterol, salbutamol, and terbutalin; bronchodilators such as carbuterol, dyphillin, etophyllin, fenoterol, pirbuterol, rimiterol and terbutalin; cardiotonics such as digitoxin, dobutamin, etilefrin, and prenalterol; antimycotics such as amphotericin B, chlorphenesin, nystatin, and perimycin; anticoagulants such as acenocoumarol, dicoumarol, phenprocoumon, and warfarin; vasodilators such as bamethan, dipyrimadol, diprophyllin, isoxsuprin, vincamin and xantinol nicotinate; antihypocholesteremics such as compactin, eptastatin, mevinolin, and simvastatin; miscellaneous drugs such as bromperidol (antipsychotic), dithranol (psoriasis) ergotamine (migraine) ivermectin (antihelminthic), metronidazole and secnizadole (antiprotozoals), nandrolon (anabolic), propafenon and quinadine (antiarythmics), quetiapine (CNS), serotonin (neurotransmitter), and silybin (hepatic disturbance).

Representative drugs containing phosphonic acid moieties include: adefovir, alendronate, AR-C69931MX, BMS-187745, ceronapril, CGP-24592, CGP-37849, CGP-39551, CGP-40116, cidofovir, clodronate, EB-1053, etidronate, fanapanel, foscamet, fosfomycin, fosinopril, fosinoprilat, ibandronate, midafotel, neridronate, olpadronate, pamidronate, residronate, tenofovir, tiludronate, WAY-126090, YH-529, and zolendronate.

Representative drugs containing phosphoric acid moieties include: bucladesine, choline alfoscerate, citocoline, fludarabine phosphate, fosopamine, GP-668, perifosine, triciribine phosphate, and phosphate derivatives of nucleoside analogs which require phophorylation for activity, such as 3TC, acyclovir, AZT, BVDU, ddC, ddI, FMAU, FTC, ganciclovir, gemcitabine, H2G, lamivudine, penciclovir and the like.

Preferred drugs for modification to prodrugs capable of colonic absorption and incorporation into sustained release formulations include the following compounds:

analgesics and/or antiinflammatory agents selected from the group consisting of acetaminophen, buprenorphine, diclofenac, diflunisal, fenoprofen, ibuprofen, indomethacin, ketoprofen, mefenamic acid, meptazinol, morphine, oxycodone, pentazocine, pethidine, tolmetin, and tramadol;

antihypertensive agents selected from the group consisting of captopril, diltiazem, methyldopa, metoprolol, prazosin, propranolol, quinapril, sotalol, and timolol;

antibiotic agents selected from the group consisting of amoxicillin, ampicillin, aztreonam, cefaclor, cefadroxil, cefixime, cefotaxime, cefoxitin, cefpodoxime, ceftizoxime, ceftriaxone, cefuroxime, cephalexin, ciproflaxacin, clindamycin, erythromycin, imipenem, mandol, meropenem, metronidazole, and tobramycin;

antiviral agents selected from the group consisting of acyclovir, delavirdine, didanosine, foscarnet, ganciclovir, indinavir, lamivudine, nelfinavir, penciclovir, ritonavir, saquinavir, stavudine, zalcitabine, and zidovudine;

bronchodilator and or anti-asthmatic agents selected from the group consisting of salbutamol and terbutaline;

antiarrhythmic agents selected from the group consisting of mexiletine, procainamide, and tocainide;

centrally acting substances selected from the group consisting of baclofen, benserazide, bupropion, carbidopa, gabapentin, levodopa, methylphenildate, pramipexole, pregabalin, quetiapine, ropinirole, and vigabatrin;

cytostatics and metastasis inhibitors selected from the group consisting of cytarabine, decitabine, docetaxal, flutamide, gemcitabine, paclitaxel, and pentostatin; and, agents for treatment of gastrointestinal disorders selected from the group consisting of cisapride, metoclopramide, and misoprostol.

VI. Pharmaceutical Compositions and Methods of Treatment

Agents that are themselves substrates for a transporter or which are linked to conjugate moieties that are substrates for a transporter can be can be incorporated into pharmaceutical compositions. Usually, although not necessarily, such pharmaceutical compositions are designed for oral administration. Oral administration of such compositions results in uptake through the intestine via a transporter and entry into the systemic circulation. The agent or conjugate component of a pharmaceutical composition can thus be efficiently delivered to a wide range of tissues in the body.

Agents optionally linked to a conjugate moiety are combined with pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, phosphate buffered saline (PBS), Ringer's solution, dextrose solution, and Hank's solution. In addition, the pharmaceutical composition or formulation can also include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents, detergents and the like (see, e.g., Remington's Pharmaceutical Sciences, Mace Publishing Company, Philadelphia, Pa., 17th ed. (1985); for a brief review of methods for drug delivery, see, Langer, Science 249:1527-1533 (1990); each of these references is incorporated by reference in its entirety).

Pharmaceutical compositions for oral administration can be in the form of e.g., tablets, pills, powders, lozenges, sachets, cachets, elixirs, suspensions, emulsions, solutions, or syrups. Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. Preserving agents such as methyl- and propylhydroxy-benzoates; sweetening agents; and flavoring agents can also be included. Depending on the formulation, compositions can provide quick, sustained or delayed release of the active ingredient after administration to the patient. In a preferred embodiment, polymeric materials are used for oral sustained release delivery (see “Medical Applications of Controlled Release,” Langer and Wise (eds.), CRC Pres., Boca Raton, Fla. (1974); “Controlled Drug Bioavailability,” Drug Product Design and Performance, Smolen and Ball (eds.), Wiley, New York (1984); Ranger and Peppas, 1983, J Macromol. Sci. Rev. Macromol Chem. 23:61; see also Levy et al., 1985, Science 228: 190; During et al., 1989, Ann. Neurol. 25:351; Howard et al, 1989, J. Neurosurg. 71:105). Sustained release can be achieved by encapuslating conjugates within a capule, or within slow-dissolving polymers. Preferred polymers include sodium carboxymethylcellulose, hydroxypropylcellulose, hydroxypropylmethylcellulose and hydroxyethylcellulose (most preferred, hydroxypropyl methylcellulose). Other preferred cellulose ethers have been described (Alderman, Int. J. Pharm. Tech. & Prod. Mfr., 1984, 5(3) 1-9). Factors affecting drug release have been described in the art (Bamba et al., Int. J. Pharm., 1979, 2, 307).

In another embodiment, enteric-coated preparations can be used for oral sustained release administration. Preferred coating materials include polymers with a pH-dependent solubility (i.e., pH-controlled release), polymers with a slow or pH-dependent rate of swelling, dissolution or erosion (i.e., time-controlled release), polymers that are degraded by enzymes (i.e., enzyme-controlled release) and polymers that form firm layers that are destroyed by an increase in pressure (i.e., pressure-controlled release). Enteric-coated osmotic capsules designed to split apart after a timed delay and deliver substantially their entire dose at a point downstream from the low pH stomach, i.e., in the colon are particularly suitable for delayed-release compositions.

In still another embodiment, osmotic delivery systems are used for oral sustained release administration (Verma et al., Drug Dev. Ind. Pharm., 2000, 26:695-708). In a preferred embodiment, OROS™ osmotic devices are used for oral sustained release delivery devices (Theeuwes et al., U.S. Pat. No. 3,845,770; Theeuwes et al., U.S. Pat. No. 3,916,899).

Conjugates or agents can be formulated as components of beads that on dissolution or diffusion release the conjugate or agent over an extended period of hours, preferably, over a period of at least 6 hours, more preferably, over a period of at least 8 hours and most preferably, over a period of at least 12 hours. The conjugate- or agent-releasing beads may have a central composition or core comprising a conjugate and pharmaceutically acceptable vehicles, including an optional lubricant, antioxidant and buffer. The beads can be medical preparations with a diameter of about 1 to 2 mm. Individual beads can comprise doses of the conjugate, for example, doses of up to about 40 mg of conjugate. Optionally, the beads are formed of non-cross-linked materials to enhance their discharge from the gastrointestinal tract. The beads can be coated with a release rate-controlling polymer that gives a timed release profile.

The time release beads can be manufactured into a tablet for therapeutically effective conjugate administration. The beads can be made into matrix tablets by the direct compression of a plurality of beads coated with, for example, an acrylic resin and blended with excipients such as hydroxypropylmethyl cellulose. The manufacture of beads has been disclosed in the art (Lu, Int. J. Pharm., 1994, 112, 117-124; Pharmaceutical Sciences by Remington, 14^{th} ed, pp1626-1628 (1970); Fincher, J. Pharm. Sci. 1968, 57, 1825-1835 ( ); and U.S. Pat. No. 4,083,949) as has the manufacture of tablets (Pharmaceutical Sciences, by Remington, 17^{th} Ed, Ch. 90, pp1603-1625 (1985).

Alternatively, an oral sustained release pump may be used (see Langer, supra; Sefton, 1987, CRC Crit Ref Biamed Eng. 14:201; Saudek et al., 1989, N. Engl. J Med. 321:574).

Drug-releasing lipid matrices can also be used for oral sustained release administration. For example, solid microparticles of the conjugate are coated with a thin controlled release layer of a lipid (e.g., glyceryl behenate and/or glyceryl palmitostearate) as disclosed in Farah et al., U.S. Pat. No. 6,375,987 and Joachim et al., U.S. Pat. No. 6,379,700. The lipid-coated particles can optionally be compressed to form a tablet. Another controlled release lipid-based matrix material which is suitable for sustained release oral administration comprises polyglycolized glycerides as disclosed in Roussin et al., U.S. Pat. No. 6,171,615.

Conjugate-releasing waxes can also be used for oral sustained release administration. Examples of suitable sustained conjugate-releasing waxes are disclosed in Cain et al., U.S. Pat. No. 3,402,240 (carnauba wax, candelilla wax, esparto wax and ouricury wax); Shtohryn et al. U.S. Pat. No. 4,820,523 (hydrogenated vegetable oil, bees wax, carnauba wax, paraffin, candelillia, ozokerite and mixtures thereof); and Walters, U.S. Pat. No. 4,421,736 (mixture of paraffin and castor wax).

In a further variation, a controlled-release system can be placed in proximity of a drug target, thus requiring only a fraction of the systemic dose (see, e.g., Goodson, in “Medical Applications of Controlled Release,” supra, vol. 2, pp. 115-138 (1984)). Other controlled-release systems discussed in Langer, 1990, Science 249:1527-1533 may also be used.

In some compositions, the dosage form comprises a conjugate coated on a polymer substrate. The polymer can be an erodible, or a non-erodible polymer. The coated substrate may be folded onto itself to provide a bilayer polymer drug dosage form. For example conjugate can be coated onto a polymer such as a polypeptide, collagen, gelatin, polyvinyl alcohol, polyorthoester, polyacetyl, or a polyorthocarbonate and the coated polymer folded onto itself to provide a bilaminated dosage form. In operation, the bioerodible dosage form erodes at a controlled rate to dispense the conjugate over a sustained release period. Representative biodegradable polymer comprise a member selected from the group consisting of biodegradable poly(amides), poly (amino acids), poly(esters), poly(lactic acid), poly(glycolic acid), poly(carbohydrate), poly(orthoester), poly (orthocarbonate), poly(acetyl), poly(anhydrides), biodegradable poly(dehydropyrans), and poly(dioxinones) which are known in the art (Rosoff, Controlled Release of Drugs, Chpt. 2, pp. 53-95 (1989); and in U.S. Pat. Nos. 3,811,444; 3,962,414; 4,066,747, 4,070,347; 4,079,038; and 4,093,709).

In some compositions, the dosage form comprises a conjugate loaded into a polymer that releases the conjugate by diffusion through a polymer, or by flux through pores or by rupture of a polymer matrix. The drug delivery polymeric dosage form comprises a concentration of 10 mg to 2500 mg homogenously contained in or on a polymer. The dosage form comprises at least one exposed surface at the beginning of dose delivery. The non-exposed surface, when present, is coated with a pharmaceutically acceptable material impermeable to the passage of the conjugate. The dosage form may be manufactured by procedures known in the art. An example of providing a dosage form comprises blending a pharmaceutically acceptable carrier like polyethylene glycol, with a known dose of conjugate at an elevated temperature, like 37° C., and adding it to a Silastic™ medical grade elastomer with a cross-linking agent, for example, octanoate, followed by casting in a mold. The step is repeated for each optional successive layer. The system is allowed to set for 1 hour, to provide the dosage form. Representative polymers for manufacturing the dosage form comprise a member selected from the group consisting of olefin, and vinyl polymers, addition polymers, condensation polymers, carbohydrate polymers, and silicon polymers as represented by polyethylene, polypropylene, polyvinylacetate, polymethylacrylate, polyisobutylmethacrylate, polyalginate, polyamide and polysilicone. The polymers and procedures for manufacturing them have been described in the art (Coleman et al., Polymers 1990, 31, 1187-1231; Roerdink et al., Drug Carrier Systems 1989, 9, 57-10.; Leong et al., Adv. Drug Delivery Rev. 1987, 1, 199-233; Roff et al., Handbook of Common Polymers 1971, CRC Press; U.S. Pat. No. 3,992,518).

In some compositions, the dosage from comprises a plurality of tiny pills. The tiny time-released pills provide a number of individual doses for providing various time doses for acheiving a sustained-release conjugate delivery profile over an extended period of time up to 24 hours. The matrix comprises a hydrophilic polymer selected from the group consisting of a polysaccharide, agar, agarose, natural gum, alkali alginate including sodium alginate, carrageenan, fucoidan, furcellaran, laminaran, hypnea, gum arabic, gum ghatti, gum karaya, gum tragacanth, locust bean gum, pectin, amylopectin, gelatin, and a hydrophilic colloid. The hydrophilic matrix comprises a plurality of 4 to 50 tiny pills, each tiny pill comprise a dose population of from 10 ng, 0.5 mg, 1 mg, 1.2 mg, 1.4 mg, 1.6 mg, 5.0 mg etc. The tiny pills comprise a release rate controlling wall of 0.001 up to 10 mm thickness to provide for the timed release of conjugate. Representative wall forming materials include a triglyceryl ester selected from the group consisting of glyceryl tristearate, glyceryl monostearate, glyceryl dipalmitate, glyceryl laureate, glyceryl didecenoate and glyceryl tridenoate. Other wall forming materials comprise polyvinyl acetate, phthalate, methylcellulose phthalate and microporous olefins. Procedures for manufacturing tiny pills are disclosed in U.S. Pat. Nos. 4,434,153; 4,721,613; 4,853,229; 2,996,431; 3,139,383 and 4,752,470.

In some compositions, the dosage form comprises an osmotic dosage form, which comprises a semipermeable wall that surrounds a therapeutic composition comprising the conjugate. In use within a patient, the osmotic dosage form comprising a homogenous composition imbibes fluid through the semipermeable wall into the dosage form in response to the concentration gradient across the semipermeable wall. The therapeutic composition in the dosage form develops osmotic energy that causes the therapeutic composition to be administered through an exit from the dosage form over a prolonged period of time up to 24 hours (or even in some cases up to 30 hours) to provide controlled and sustained conjugate release. These delivery platforms can provide an essentially zero order delivery profile as opposed to the spiked profiles of immediate release formulations.

In some compositions, the dosage form comprises another osmotic dosage form comprising a wall surrounding a compartment, the wall comprising a semipermeable polymeric composition permeable to the passage of fluid and substantially impermeable to the passage of conjugate present in the compartment, a conjugate-containing layer composition in the compartment, a hydrogel push layer composition in the compartment comprising an osmotic formulation for imbibing and absorbing fluid for expanding in size for pushing the conjugate composition layer from the dosage form, and at least one passageway in the wall for releasing the conjugate composition. The method delivers the conjugate by imbibing fluid through the semipermeable wall at a fluid imbibing rate determined by the permeability of the semipermeable wall and the osmotic pressure across the semipermeable wall causing the push layer to expand, thereby delivering the conjugate from the dosage form through the exit passageway to a patient over a prolonged period of time (up to 24 or even 30 hours). The hydrogel layer composition may comprise 10 mg to 1000 mg of a hydrogel such as a member selected from the group consisting of a polyalkylene oxide of 1,000,000 to 8,000,000 which are selected from the group consisting of a polyethylene oxide of 1,000,000 weight-average molecular weight, a polyethylene oxide of 2,000,000 molecular weight, a polyethylene oxide of 4,000,000 molecular weight, a polyethylene oxide of 5,000,000 molecular weight, a polyethylene oxide of 7,000,000 molecular weight and a polypropylene oxide of the 1,000,000 to 8,000,000 weight-average molecular weight; or 10 mg to 1000 mg of an alkali carboxymethylcellulose of 10,000 to 6,000,000 weight average molecular weight, such as sodium carboxymethylcellulose or potassium carboxymethylcellulose. The hydrogel expansion layer comprises 0.0 mg to 350 mg, in present manufacture; 0.1 mg to 250 mg of a hydroxyalkylcellulose of 7,500 to 4,500,00 weight-average molecular weight (e.g., hydroxymethylcellulose, hydroxyethylcellulose, hydroxypropylcellulose, hydroxybutylcellulose or hydroxypentylcellulose) in present manufacture; 1 mg to 50 mg of an osmotic agent selected from the group consisting of sodium chloride, potassium chloride, potassium acid phosphate, tartaric acid, citric acid, raffinose, magnesium sulfate, magnesium chloride, urea, inositol, sucrose, glucose and sorbitol; 0 to 5 mg of a colorant, such as ferric oxide; 0 mg to 30 mg, in a present manufacture, 0.1 mg to 30 mg of a hydroxypropylalkylcellulose of 9,000 to 225,000 average-number molecular weight, selected from the group consisting of hydroxypropylethylcellulose, hydroxypropypentylcellulose, hydroxypropylmethylcellulose, and hydropropylbutylcellulose; 0.00 to 1.5 mg of an antioxidant selected from the group consisting of ascorbic acid, butylated hydroxyanisole, butylatedhydroxyquinone, butylhydroxyanisol, hydroxycomarin, butylated hydroxytoluene, cephalm, ethyl gallate, propyl gallate, octyl gallate, lauryl gallate, propyl-hydroxybenzoate, trihydroxybutylrophenone, dimethylphenol, dibutylphenol, vitamin E, lecithin and ethanolamine; and 0.0 mg to 7 mg of a lubricant selected from the group consisting of calcium stearate, magnesium stearate, zinc stearate, magnesium oleate, calcium palmitate, sodium suberate, potassium laureate, salts of fatty acids, salts of alicyclic acids, salts of aromatic acids, stearic acid, oleic acid, palmitic acid, a mixture of a salt of a fatty, alicyclic or aromatic acid, and a fatty, alicyclic, or aromatic acid.

In the osmotic dosage forms, the semipermeable wall comprises a composition that is permeable to the passage of fluid and impermeable to the passage of conjugate. The wall is nontoxic and comprises a polymer selected from the group consisting of a cellulose acylate, cellulose diacylate, cellulose triacylate, cellulose acetate, cellulose diacetate and cellulose triacetate. The wall comprises 75 wt % (weight percent) to 100 wt % of the cellulosic wall-forming polymer; or, the wall can comprise additionally 0.01 wt % to 80 wt % of polyethylene glycol, or 1 wt % to 25 wt % of a cellulose ether selected from the group consisting of hydroxypropylcellulose or a hydroxypropylalkylcellulose such as hydroxypropylmethylcellulose. The total weight percent of all components comprising the wall is equal to 100 wt %. The internal compartment comprises the conjugate-containing composition alone or in layered position with an expandable hydrogel composition. The expandable hydrogel composition in the compartment increases in dimension by imbibing the fluid through the semipermeable wall, causing the hydrogel to expand and occupy space in the compartment, whereby the drug composition is pushed from the dosage form. The therapeutic layer and the expandable layer act together during the operation of the dosage form for the release of conjugate to a patient over time. The dosage form comprises a passageway in the wall that connects the exterior of the dosage form with the internal compartment. The osmotic powered dosage form provided by the invention delivers conjugate from the dosage form to the patient at a zero order rate of release over a period of up to about 24 hours.

The expression “passageway” as used herein comprises means and methods suitable for the metered release of the conjugate from the compartment of the dosage form. The exit means comprises at least one passageway, including orifice, bore, aperture, pore, porous element, hollow fiber, capillary tube, channel, porous overlay, or porous element that provides for the osmotic controlled release of conjugate. The passageway includes a material that erodes or is leached from the wall in a fluid environment of use to produce at least one controlled-release dimensioned passageway. Representative materials suitable for forming a passageway, or a multiplicity of passageways comprise a leachable poly(glycolic) acid or poly(lactic) acid polymer in the wall, a gelatinous filament, poly(vinyl alcohol), leach-able polysaccharides, salts, and oxides. A pore passageway, or more than one pore passageway, can be formed by leaching a leachable compound, such as sorbitol, from the wall. The passageway possesses controlled-release dimensions, such as round, triangular, square and elliptical, for the metered release of conjugate from the dosage form. The dosage form can be constructed with one or more passageways in spaced apart relationship on a single surface or on more than one surface of the wall. The expression “fluid environment” denotes an aqueous or biological fluid as in a human patient, including the gastrointestinal tract. Passageways and equipment for forming passageways are disclosed in U.S. Pat. Nos. 3,845,770; 3,916,899; 4,063,064; 4,088,864 and 4,816,263. Passageways formed by leaching are disclosed in U.S. Pat. Nos. 4,200,098 and 4,285,987.For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 mg to about 2 g of the active agent.

The compositions can be administered for prophylactic and/or therapeutic treatments. A therapeutic amount is an amount sufficient to remedy a disease state or symptoms, or otherwise prevent, hinder, retard, or reverse the progression of disease or any other undesirable symptoms in any way whatsoever. In prophylactic applications, compositions are administered to a patient susceptible to or otherwise at risk of a particular disease or infection. Hence, a “prophylactically effective” is an amount sufficient to prevent, hinder or retard a disease state or its symptoms. In either instance, the precise amount of compound contained in the composition depends on the patient's state of health and weight.

An appropriate dosage of the pharmaceutical composition is readily determined according to any one of several well-established protocols. For example, animal studies (e.g., mice, rats) are commonly used to determine the maximal tolerable dose of the bioactive agent per kilogram of weight. In general, at least one of the animal species tested is mammalian. The results from the animal studies can be extrapolated to determine doses for use in other species, such as humans for example.

The components of pharmaceutical compositions are preferably of high purity and are substantially free of potentially harmful contaminants (e.g., at least National Food (NF) grade, generally at least analytical grade, and more typically at least pharmaceutical grade). To the extent that a given compound must be synthesized prior to use, the resulting product is typically substantially free of any potentially toxic agents, particularly any endotoxins, which may be present during the synthesis or purification process. Compositions for oral administration need are usually made under GMP conditions.

### EXAMPLES

I. PCR Analysis of Transporter Expression.

Oligonucleotide primers were designed to amplify unique transporter DNA sequences. All primers had annealing temperatures above 55° C. and products were sequenced to verify specificity. Transporter expression was quantitated by PCR (polymerase chain reaction) amplification using real-time PCR (Cepheid Smartcycler PCR instrument; MJ Research Opticon PCR instrument; and Perkin-Elmer SYBR-green reagents; all protocols per manufacturers specifications). Single-stranded cDNA was prepared from human mRNA (purchased from Clontech, BioChain, and Stratagene) using Thermoscript (Stratagene) reverse transcriptase kit. Real-time PCR was performed using the primer sets listed above to amplify fragments of the transporter mRNAs. In addition, total mRNA abundance was normalized by measurement of GAPDH or beta actin levels in each tissue. Transcript abundance was measured by determining the threshold cycle and calculating transcript number using a calibration factor derived from amplification of known plasmid copy numbers. In order to compare different tissues, all data is expressed as fraction of GAPDH or beta actin transcript levels.

II. Synthesis of Conjugates

1. Preparation of Pivaloxymethyl Gabapentin Carbamate

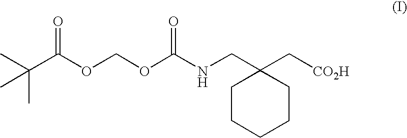

p-Nitrophenol (4.2 g, 30 mmol) was dissolved in anhydrous tetrahydrofuran (300 mL) and stirred vigorously. To this solution was added chloromethyl chloroformate (2.7 mL, 30 mmol) followed by triethylamine (4.2 mL, 30 mmol). A white precipitate (triethylamine hydrochloride) was formed immediately and the reaction stirred for 30 minutes. The precipitate was then removed by filtration, and the volatile organic components removed under reduced pressure to yield a yellow or yellow-brown oil. This residue was redissolved in dichloromethane (250 mL) and washed twice with saturated aqueous sodium carbonate (200 mL) to remove unreacted p-nitrophenol, once with 1N HCl (200 mL), once with saturated sodium bicarbonate and then finally with saturated sodium chloride. The organic layer was dried over anhydrous sodium sulfate, filtered and then dried under reduced pressure to yield analytically pure chloromethyl p-nitrophenyl carbonate as a pale yellow oil in excellent yield (90-99%). The compound was unstable to LC-MS. ¹H NMR (CDCl₃, 400 MHz): 5.86 (s, 2H), 7.44 (d, J=9 Hz, 2H), 8.33 (d, J=9 Hz, 2H).

Chloromethyl p-nitrophenyl carbonate (4.7 g, 20 mmol) was dissolved in anhydrous acetone (250 mL). To this was added sodium iodide (4.5 g, 30 mmol) and anhydrous sodium bicarbonate (3.4 g, 40 mmol). The reaction was heated to 60° C. with vigorous stirring for 12-24 h, during which time the progress of the reaction was followed by ¹H NMR. Upon completion, the solid materials were removed by filtration and the solvent was removed under reduced pressure to yield a yellow oil. This residue was redissolved in dichloromethane (200 mL) and washed twice with saturated aqueous sodium carbonate (200 mL) followed by water (100 mL). The organic layer was then dried over anhydrous sodium sulfate, filtered and the volatile components removed under reduced pressure to yield a pale yellow oil that may solidify upon standing to yield dark yellow crystals of iodomethyl p-nitrophenyl carbonate. The compound was found to be unstable to LC-MS. ¹H NMR (CDCl₃, 400 MHz): 6.06 (s, 2H), 7.42 (d, J=9 Hz, 2H), 8.30 (d, J=9 Hz, 2H). ¹³C NMR (CDCl₃, 100 MHz): 155.1, 151.0, 146.0, 125.8, 125.7, 121.9, 33.5.

Pivalic acid (1.0 g, 10 mmol) was dissolved in water (20 ml). To this was added silver oxide (1.6 g, 7 mmol). The mixture was shaken at 60° C. for 4 h, yielding a copious gray precipitate. The mixture was poured into of distilled water (350 ml) and brought to a boil to dissolve the grey material. The hot solution was then filtered to remove unreacted silver oxide. The water was removed under reduced pressure to yield a pale white or silvery white solid. This material was found to react rapidly with sodium iodide in water to form a pale yellow precipitate, indicating the presence of silver ions (yield: 40-80%).

Iodomethyl p-nitrophenyl carbonate (325 mg, 1.0 mmol) was dissolved in anhydrous toluene (15 ml). Silver pivaloate (270 mg, 1.3 mmol) was added and the reaction stirred or shaken at 60° C. for 12 h. The reaction mixture was filtered to remove excess solid and then poured into dichloromethane (100 ml), and washed twice with saturated sodium carbonate (100 ml), once with 1N HCl (100 ml), once with saturated sodium bicarbonate solution (100 ml) and once with saturated sodium chloride (50 ml). The organic layer was dried over anhydrous sodium sulfate, filtered and then the volatile components were removed under reduced pressure to yield a yellow oil. This was purified by silica gel chromatography (8:1 hexanes: EtOAc) to yield pivaloxymethyl p-nitrophenyl carbonate as a pale yellow oil.

¹H NMR (CDCl₃, 400 MHz): 1.25 (s, 9H), 5.88 (s, 2H), 7.40 (d, J=9 Hz, 2H), 8.29 (d, J=9 Hz, 2H). ¹³C NMR (CDCl₃, 100 MHz): 177.0, 155.3, 151.6, 145.8, 125.6, 121.9, 83.1, 39.1, 27.0.

Finely ground gabapentin hydrochloride (100 mg, 0.5 mmol) was placed in a round bottom flask with anhydrous dichloromethane (25 mL) under nitrogen. Trimethylsilyl chloride (750 μL, 0.6 mmol) was added, followed by triethylamine (1.4 mL, 1.0 mmol) and the reaction allowed to stir for 15-30 minutes until the gabapentin had largely dissolved. Pivaloxymethyl p-nitrophenyl carbonate (150 mg, 0.5 mmol) was then added and the reaction stirred at room temperature for 18 h, until found to be complete (as monitored by LC-MS). The reaction was poured into ethyl acetate (200 mL) and washed twice with 1N HCl. The organic layer was then dried under reduced pressure and purified by reverse phase HPLC using a ion spray mass spectrometer to identify the product peak. The product containing fractions were pooled, frozen to −78° C. and lyophilized to yield a clear oil, which was gabapentin pivaloxymethyl carbamate (Compound I). MS (ESI) m/z 328.36 (M−H⁻), 330.32 (M+H⁺), 352.33 (M+Na⁺). ¹H NMR (CDCl₃, 400 MHz): 1.21 (s, 9H), 1.3-1.5 (m, 10H), 2.32 (s, 2H), 3.26 (s, 2H), 5.33 (m, 1H), 5.73 (s, 2H). ¹³C NMR (CDCl₃, 400 MHz): 178.0, 176.8, 155.9, 80.6, 39.2, 38.2, 34.3, 27.3, 26.2, 21.7.

2. Preparation of Gabapentin Phenylacetoxymethyl Carbamate

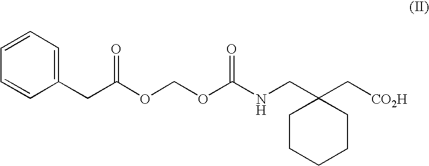

Following the above protocol and substituting phenylacetic acid for pivalic acid, gabapentin phenylacetoxymethyl carbamate (Compound II) was obtained. MS (ESI) m/z 362.4 (M−H⁻), 364.4 (M+H⁺).

3. Preparation of Gabapentin Benzoyloxymethyl Carbamate

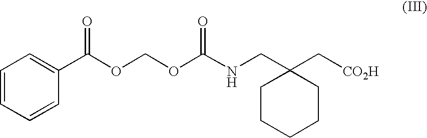

Following the above protocol and substituting benzoic acid for pivalic acid, gabapentin benzoyloxymethyl carbamate (Compound III) was obtained. MS (ESI) m/z 348.4 (M−H⁻), 350.4 (M+H⁺).

4. Preparation of Gabapentin Acetoxyethyl Carbamate

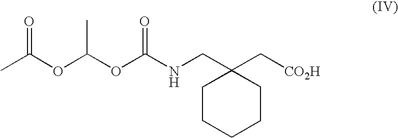

To an ice cold reaction mixture containing p-nitrophenol (1.39 g, 10 mmol) and pyridine (0.81 g, 10 mmol) in dichloromethane (60 mL) was added 1-chloroethyl chloroformate (1.2 mL, 11 mmol). The mixture was stirred at 0° C. for 30 min and then at room temperature for 1 h. After evaporation of the solvent under reduced pressure, the residue was dissolved in ether and washed with water, 0.5% (v/v) aqueous NaHCO₃, and water again. The ether layer was dried over Na₂SO₄ and evaporated under reduced pressure to give an off-white solid (2.4 g, 97%), which was 1-chloroethyl-p-nitrophenyl carbonate. ¹H NMR (CDCl₃): 1.93 (d, 3H, CH₃), 6.55 (q, 1H, CH), 7.42 (d, 2H, aromatic), 8.28 (d, 2H, aromatic).

A mixture containing 1-chloroethyl-p-nitrophenyl carbonate (0.5 g, 2 mmol) and NaI (0.6 g, 4 mmol) in dry acetone was stirred for 3 h at 40° C., followed by filtration, and washing with ether. The filtrate was evaporated under reduced pressure and the resulting 1-iodoethyl-p-nitrophenyl carbonate (480 mg, 72%) was used as is.

A mixture containing NaHCO₃ (0.336 g, 4 mmol), tetrabutylammonium bisulfate (0.68 g, 2 mmol), acetic acid (0.122 g, 2 mmol), water (5 mL), and dichloromethane (10 mL) was stirred at room temperature for 1 h. A solution of 1-iodoethyl-p-nitrophenyl carbonate (0.674 g, 2 mmol) in dichloromethane (10 mL) was added and the reaction mixture stirred for 16 h. The organic phase was separated and washed with water, dried over Na₂SO₄, and evaporated under reduced pressure. Chromatography of the resulting residue on silica gel, eluting with hexane:ethyl acetate (95:5), gave pure α-acetoxyethyl-p-nitrophenyl carbonate product (0.11 g, 21%). ¹H NMR (CDCl₃): 1.58 (d, 3H, CH₃), 2.11 (s, 3H, Ac), 6.84 (q, 1H, CH), 7.39 (d, 2H, aromatic), 8.26 (d, 2H, aromatic).

Alternatively, α-acetoxyethyl-p-nitrophenyl carbonate could be made directly from 1-chloroethyl-p-nitrophenyl carbonate by the following procedure. A mixture of 1-chloroethyl-p-nitrophenyl carbonate (0.5 g, 2 mmol) and mercuric acetate (1.5 g, 4.4 mmol) in acetic acid (15 mL) was stirred at room temperature for 24 h. After removal of acetic acid under reduced pressure, the residue was dissolved in ether and washed with water, 0.5% (v/v) aqueous NaHCO₃, and water again. The ether layer was dried over Na₂SO₄, and concentrated to dryness. Chromatography of the resulting residue on silica gel, eluting with hexane:ethyl acetate (95:5), gave pure carbonate product (0.45 g, 84%).

To a mixture containing gabapentin (633 mg, 3.7 mmol) and triethylamine (1.03 mL, 7.4 mmol) in dichloromethane (20 mL) was added trimethylchlorosilane (0.93 mL, 7.4 mmol) and the mixture stirred until clear. A solution containing α-acetoxyethyl-p-nitrophenyl carbonate (1 g, 3.7 mmol) in dichloromethane (10 mL) was added and stirred for 30 min. The reaction mixture was washed with saturated aqueous NaHCO₃ (20 mL) and the organic phase separated. The aqueous layer was further extracted with ether (3×10 mL) and the combined organic phases were dried over MgSO4 then concentrated in vacuo. Chromatography of the resulting residue on silica gel, eluting with hexane:ethyl acetate (4:1) gave the desired pure gabapentin acetoxyethyl carbamate (Compound IV) (700 mg, 63%).

¹H NMR (CDCl₃): 1.27-1.60 (m, 10H cyclohexyl), 1.55 (d, 3H, CH₃), 2.08 (s, 3H, Ac), 2.38 (s, 2H, CH₂), 3.25 (m, 2H, CH₂), 5.31 (t, 1H, OH), 6.81 (q, 1H, CH); MS (ESI) m/z 302.2 (M+H⁺). The acid form was quantitatively converted to the corresponding sodium salt by dissolution in water (5 mL), addition of an equimolar quantity of 0.5 N NaHCO₃, followed by lyophilization.

5. Preparation of α-Aminoisobutyryl Gabapentin

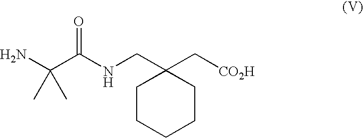

To a 40 mL vial was added N-Boc-α-aminoisobutyric acid (5 mmol), dicyclohexylcarbodiimide (1.24 g, 6 mmol), N-hydroxysuccinimide (0.7 g, 6 mmol), and acetonitrile (20 mL). The reaction mixture was shaken at 22-25° C. for 4 h. The precipitated dicyclohexylurea was removed by filtration. To the filtrate was added an aqueous solution (30 mL) of gabapentin hydrochloride (1.04 g, 6 mmol), and sodium hydroxide (0.4 g, 10 mmol). The reaction was stirred at 22-25 C for 16 h. The reaction mixture was diluted with ethyl acetate (100 mL) and washed with 0.5 M aqueous citric acid (2×100 mL) and water (2×100 mL). The organic phase was separated, dried (MgSO₄), filtered and concentrated under reduced pressure. The residue was dissolved in trifluoroacetic acid (40 mL) and allowed to stand at 22-25° C. for 2 h. The solvent was removed under reduced pressure. The residue was dissolved in water (4 mL) and filtered through a 0.25 μm nylon membrane filter prior to purification by preparative HPLC (Phenomenex 250×21.2 mm, 5 μm LUNA C18 column, 100% water for 5 minutes, then 0-60% acetonitrile in water with 0.05% trifluoroacetatic acid over 20 minutes at 20 mL/min). The pure fractions were combined and the solvent was removed under reduced pressure to afford the product a-aminoisobutyryl gabapentin (Compound V) as a white solid (yield˜70%).

MS (ESI) m/z 255.26 (M−H⁻), 257.28 (M+H⁺).

III. Analysis of Transport of Naturally Expressed Transporters in HEK Cells

Although HEK's are a kidney derived cell line, they express some of the same transporters as the colon and can be used as a preliminary screen to identify substrates of colon-expressed transporters.

pH Assay Protocol:

Cells: HEK peak

Buffers:

Buffer 1

1 mM CaCl₂

1 mM MgCl₂

150 mM NaCl

3 mM KCl

1 mM NaH₂PO₄

5 mM Glucose

50 mM 4-(2-hydroxyethyl)-1-piperazineethanesulfonic acid (HEPES)

pH 7.4

Buffer 2

As above, but substitute 50 mM 2-[(2-amino-2-oxoethyl)-amino]ethanesulfonic acid (ACES) for 50 mM HEPES.

pH 6.7

Buffer 3

120 mM KCl

30 mM NaCl

0.2 mM MgSO₄

1 mM CaCl

1 mM NHPO₄

5 mM Glucose

10 mM HEPES

10 mM piperazine-1,4-bis(2-ethane sulfonic acid) (PIPES)

Adjusted to different pH's

Cells were seeded at 100,000/well in 96 black, clear bottom plate overnight and washed twice in 100 μL buffer I at room temperature.

Cells were loaded with 11M 2,7-bis-(2-carboxyethyl)-5-carboxyfluoroscein acetoxymethyl ester (BCECF AM) (resuspended in 50:50 dimethylsulfoxide:Pluronic™ surfactant mixture) in buffer 1 for 15 min. at 37° C. at 50 μL/well.

The rest of the protocol was performed at room temperature.

Cells were washed twice in buffer 2 at 50 μL/well. A first reading was taken in FLEX station in the buffer 2 at two sets of fluorescence excitation/emission wavelengths, 440/535 and 490/535, with 50 μL buffer/well. Phloretin was added to the wells at 0.5 mM in 50 μL/well in buffer 2, followed by a 5 min incubation at room temperature. A second reading was taken in FLEX station at above settings (T0). Substrates were then added at two times the final concentration at 50 μL/well in buffer 2. A third reading was taken in FLEX station at above settings (T1). The assay solutions were then removed. Calibration curves were generated with buffer 3 at pH 9.7; 8.4; 7.4; 7.0, 6.5; 6.0; 5.5; and 5.0 with 10 μM nigericin.

Calculations:

For each well, values for A, B and C were calculated using the T0 and T1 data and the following equations:

A=measured fluorescence at excitation/emission wavelengths 440/535−background

B=measured fluorescence at excitation/emission wavelengths 490/535−background

C=B/A.

The C values for the T0 and T1 data were used to determine the percent decrease in fluorescence at T1 relative to T0. These values were then normalized to T0 and the data was expressed as a percent of specific lactate response.

The normalized percent decrease in C was then calculated and plotted vs. pH.

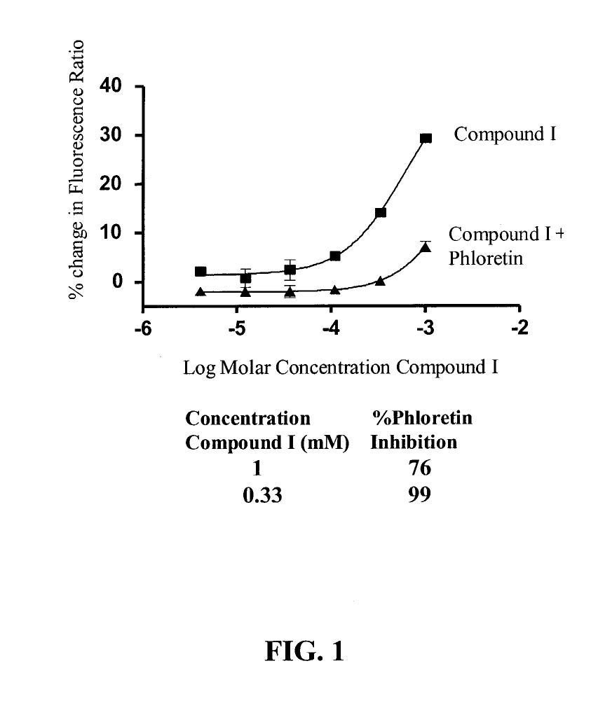

FIG. 1 shows uptake of Compound I by HEK cells in the presence and absence of a transporter inhibitor phloretin. It can be seen that phloretin substantially inhibits uptake of Compound I indicating that the uptake is transporter mediated. MCT transporters are likely candidates because they have appropriate substrate specificity and are expressed in HEK cells (and the colon).

IV. In Vitro Compound Transport Assays with PEPT1 and PEPT2-Expressing Cell Lines

(a) Inhibition of Radiolabeled Gly-Sar Uptake

Rat and human PEPT1 and PEPT2 expressing CHO cell lines were prepared as described in PCT Application WO01/20331. Gabapentin-containing dipeptides were evaluated for interaction with the peptide transporters using a radiolabeled substrate uptake assay in a competitive inhibition format, as described in PCT Application WO01/20331. Transport-induced currents were also measured in Xenopus oocytes transfected with rat and human PEPT1 and PEPT2.

(b) Analysis of Electrogenic Transport in Xenopus Oocytes

RNA preparation: Rat and human PEPT1 and PEPT2 transporter cDNAs were subcloned into a modified pGEM plasmid that contains 5′ and 3′ untranslated sequences from the Xenopus β-actin gene. These sequences increase RNA stability and protein expression. Plasmid cDNA was linearized and used as template for in vitro transcription (Epicentre Technologies transcription kit, 4:1 methylated:non-methylated guanosine triphosphate(GTP)).

Xenopus oocyte isolation. Xenopus laevis frogs were anesthetized by immersion in Tricaine (1.5 g/mL in deionized water) for 15 min. Oocytes were removed and digested in frog Ringer's solution (90 mM NaCl, 2 mM KCl, 1 mM MgCl₂, 10 mM Na HEPES, pH 7.45, no CaCl₂) with 1 mg/mL collagenase (Worthington Type 3) for 80-100 min with shaking. The oocytes were washed 6 times, and the buffer changed to frog Ringer's solution containing CaCl₂ (1.8 mM). Remaining follicle cells were removed if necessary. Cells were incubated at 16° C., and each oocyte injected with 10-20 μg RNA in 45 μL solution.

Electrophysiology measurements. Transport currents were measured 2-14 days after injection, using a standard two-electrode electrophysiology set-up (Geneclamp 500 amplifier, Digidata 1320/PCLAMP software and ADInstruments hardware and software were used for signal acquisition). Electrodes (2-4 mΩ) were microfabricated using a Sutter Instrument puller and filled with 3M KCl. The bath was directly grounded (transporter currents were less than 0.3 μA). Bath flow was controlled by an automated perfusion system (ALA Scientific Instruments, solenoid valves).

For transporter pharmacology, oocytes were clamped at −60 to −90 mV, and continuous current measurements acquired using PowerLab Software and an ADInstruments digitizer. Current signals were lowpass filtered at 20 Hz and acquired at 4-8 Hz. All bath and drug-containing solutions were frog Ringers solution containing CaCl₂. Drugs were applied for 10-30 seconds until the induced current reached a new steady-state level, followed by a control solution until baseline currents returned to levels that preceded drug application. The difference current (baseline subtracted from peak current during drug application) reflected the net movement of charge resulting from electrogenic transport and was directly proportional to transport rate. Recordings were made from a single oocyte for up to 60 min, enabling 30-40 separate compounds to be tested per oocyte. Compound-induced currents were saturable and gave half-maximal values at substrate concentrations comparable to radiolabel competition experiments. To compare results between oocytes expressing different levels of transport activity, a saturating concentration of glycyl-sarcosine (1 mM) was used as a common reference to normalize results from test compounds. Using this normalization procedure Vₘₐₓ (i.e. maximal induced current) for different compounds tested on different oocytes could be compared.

It was found that Compound V, at a concentration of 1 mM, was transported with a Vmax of 50% that of the reference substrate Gly-Sar in oocytes transfected with rat PEPT1, and with a Vmax of 66% that of the reference substrate Gly-Sar in oocytes transfected with human PEPT1. The Vmax of Compound V was<5% of Gly-Sar in the presence of the PEPT inhibitor Lys(ε-Dansyl)-Leu when tested on either rat or human PEPT transfected oocytes.

V. Experimental Methods for Measurement of SMVT and ATBO+ Transport Activity

ATBO+ is a broad-specificity amino acid transporter expressed in the colon and lung. ATBO+ belongs to the Na/Cl coupled gamma aminobutyric acid (GABA) and glycine transporter family. Among the 20 genetically encoded amino acids this transporter transports all neutral and positive charged amino acids, but not acidic amino acids (Asp, Glu). The SMVT transporter refers to the sodium-dependent multivitamin transporter SLC5A6, and is expressed in the human intestine, particularly the stomach, jejunum, ileum, the ileo-caecal valve, the cecum and the ascending colon.

1. Transporter Cloning

The complete open reading frame of human ATBO+(SLC6A14) and SMVT (SLC5A6) were amplified from human cDNA prepared from liver or intestine mRNA. Gene-specific oligonucleotide primers were designed against Genbank sequences (AF151978 and NM-021095). Amplified PCR products were cloned into a modified version of the mammalian expression vector pcDNA3 (termed pMO) that was engineered to contain the 5′ and 3′ untranslated regions from the Xenopus beta-globin gene. All clones were completely sequenced and tested for function by transient transfection in HEK293 cells. Radiolabeled ³H glycine and ³H biotin were used to assess ATB0+ and SMVT function respectively (method below).

2. Xenopus Oocyte Expression and Electrophysiology

cRNA for oocyte expression was prepared by linearization of plasmid cDNA and in vitro transcription using T7 polymerase (Epicentre Ampliscribe kit). Xenopus oocytes were prepared and maintained as previously described (Collins et al., PNAS 13:5456-5460 (1997)) and injected with 10-30 ng RNA. Transport currents were measured 2-6 days later using two-electrode voltage-clamp (Axon Instruments). All experiments were performed using a modified oocyte Ringers solution (90 mM NaCl, 2 mM KCl, 1.8 mM CaCl₂, 1 mM MgCl₂, and 10 mM Na HEPES, pH 7.4; in Na⁺-free solutions 90 mM choline chloride was substituted for NaCl). The membrane potential of oocytes was held at −60 mV and current traces acquired using PowerLab software (AD Instruments). Full 7-concentration dose-responses were performed for each compound. Current responses at the highest concentration were normalized to the maximal glycine (3 mM for ATB0+) or biotin (0.5 mM for SMVT) elicited currents. Half-maximal concentrations were calculated using non-linear regression curve fitting software (Prism) with the Hill co-efficient fixed to 1. To ensure that currents were specific for the over-expressed transporter, all compounds were tested against uninjected oocytes. Since both ATBO+ and SMVT require Na⁺ for transport, we confirmed transport specificity by application of the compounds in a Na⁺-free solution.

3. Construction of Stable Cell Lines and IC₅₀ Measurements

Stable clones of CHOK1 cells were obtained by electroporation, selection in G418, and single cell sorting using FACS (flow-activated cell sorting, Cytomation). Stable clones expressing ATBO+ or SMVT were identified by enhanced uptake of radiolabeled substrates. For cell uptake studies, stable CHOK1 clones were seeded into polylysine coated 96-well microtitre plates and grown for 2-3 days. Cells were incubated with experimental solutions (combinations of radiolabeled and unlabeled compounds) for 30 minutes at room temperature, washed four times, and lysed in scintillation solution. Accumulation of radiolabeled molecules was measured in a microtitre scintillation plate reader (Perkin Elmer). Inhibition constants (IC₅₀s) were calculated using curve-fitting software (Prism).

4. Measurement of Uptake by LC/MS/MS

Uptake of unlabeled compounds was measured in cells stably expressing SMVT or ATB0+. Cells were plated at a density of 100,000 cells/well in polylysine coated 96-well microtitre plates and assayed 24-48 hours after plating. Test compounds (0.1 to 3 mM final concentration) were added to a Hanks buffered saline solution (HBSS) and 0.1 ml of test solutions were added to each well. Cells were allowed to take up test compounds for 20-60 minutes. Test solutions were aspirated and cells washed 4 times with ice-cold HBSS. Cells were then lysed in a 50% ethanol solution (0.04 mL/well) and sonicated 10 minutes. Following sonication, 0.03 mL of lysate was removed and the concentration of test compounds determined by analytical LC/MS/MS. Transporter specific uptake was determined by comparison with control cells lacking transporter expression or transport in the absence of Na⁺.

5. Results

In vitro transport data for selected compounds on hSMVT-expressing cells

| | IC₅₀ | % Max. |
|---|---|---|
| COMPOUND | (μM) | (Biotin) |
| Gabapentin | >500 | 0 |
| Compound I | 450 | 21 |
| Compound IV | 80 | ND |
| Compound V | 320 | 36 |

IC₅₀ data from radiolabeled competition assay in SMVT-expressing CHO cells
% Max response (relative to biotin) from transporter-expressing oocytes at a test compound concentration of 0.5 mM.
ND-not determined

VI. Caco-2 General Screening Protocol

Caco-2 cells are derived from the human colon and naturally express a number of colon-expressed transporters. The cells can be used to screen agents or conjugates for capacity to be transported by a colon expressed transporter. By screening agents or conjugates in the presence and absence of the specific PEPT1 and PEPT2 inhibitor Lys(ε-Dansyl)-Leu, one can determine whether PEPT1 and/or PEPT2 is a transporter mediating transport of the agent or conjugate. The role of PEPT1 and/or PEPT2 is shown by a decrease in transport in the presence of Lys(ε-Dansyl)-Leu.

1. Method

1. Caco-2 cells are plated in either a 12 or 24 well Transwell plate and allowed to differentiate for 19-30 days prior to screening. Day 21 cells are optimal.

2. Dilutions of test compounds with or without Lys(ε-Dansyl)-Leu are prepared in assay buffer. pH 6.0

a. Concentrations of compounds are generally 1 mM with or without 600 μM Lys(Dansyl)-leucine.

b. 20 μM Propidium Iodide added as marker.

3. Spent media is aspirated from apical and basolateral chambers. To the apical chambers, 500 μL of test compound with or without Lys(ε-Dansyl)-Leu is added (125 μL for 24 well Transwell plates).

4. In the basolateral chambers, HBSS buffer pH 7.4 is added (1.5 mL for 12 well format, 875 μL for 24 well format).

5. At each timepoint, 50 μL is sampled from basolateral chambers and transferred to a LC/MS plate (Nunc, PP round bottom).

6. After the final timepoint, the membranes are removed from the Transwell using a scalpel or razor blade. Membranes are washed in buffer to remove excess compound and placed in a 125 μL or 500 μL volume of a 50/50% methanol/water solution. Plates are sonicated for 5 min. Following sonication, plates are spun in a tabletop centrifuge at 2500 rpm for 5 min. 50 μL samples are taken and placed in the LC/MS plate.

7. The plate containing the samples are generally diluted 1:2 or 1:4 in PepT1 buffer pH 6.0.

8. Samples are frozen at −20° C. until run.

2. Results

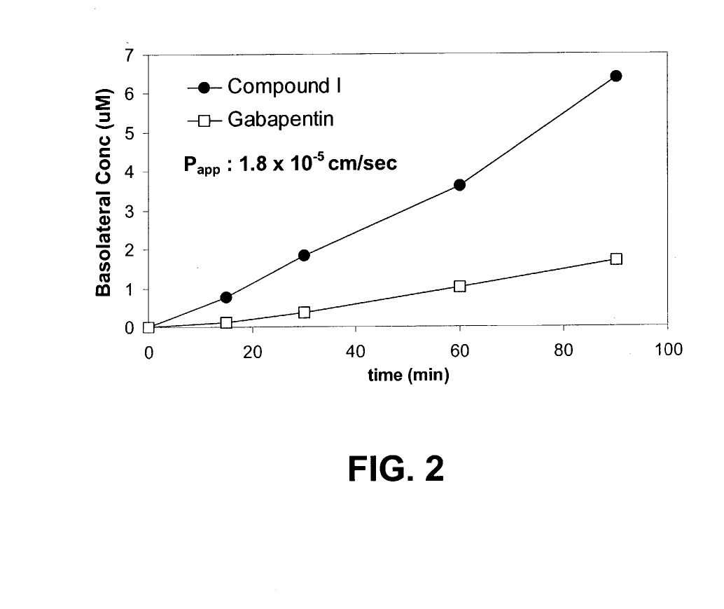

FIG. 2 compares transport of gabapentin conjugate Compound V in the presence and absence of PEPT1/PEPT2 inhibitor Lys(ε-Dansyl)-Leu. The results show that Compound V transport across Caco-2 cells is inhibited by Lys(ε-Dansyl)-Leu indicating that PEPT1 and/or PEPT2 mediate the transport. Because these transporters are expressed in the colon, Compound V can be taken up through the colon.

VII. Uptake of Compounds Through the Rat Colon

An example of a compound whose release cannot be extended by colonic administration is gabapentin. Gabapentin is administered orally, usually three to four times per day, depending on the indication. In the small intestine, the drug is absorbed by a relatively specific facilitated exchange mechanism, a transporter of large neutral amino acids. This particular transporter is present only in the small intestine, and because the residence time of materials in the small intestine is short (usually only a few hours) and rather variable, an sustained release formulation of the types described above cannot provide an effective extension of exposure to a single dose of gabapentin. As gabapentin appears not to be absorbed by non-specific passive mechanisms, and because a gabapentin-specific transporter is not present in the colon, extended colonic release is not an available option. This example shows that certain conjugates or prodrugs of gabapentin are substrates of a transport mechanism in the colon, and thus can be delivered as a sustained release formulation.

1. Administration Protocol

Rats were obtained commercially and were pre-cannulated in the both the ascending colon and the jugular vein. Animals were conscious at the time of the experiment. All animals were fasted overnight and until 4 hours post-dosing. Prodrugs were administered as a solution (in water or polyethylene glycol 400) directly into the colon via the cannula at a dose equivalent to 25 mg of gabapentin per kg. Blood samples (0.5 mL) were obtained from the jugular cannula at intervals over 8 hours and were quenched immediately by addition of acetonitrile/methanol to prevent further conversion of the prodrug. Blood samples were analyzed as described in the attached sample analysis summary.

2. Sample Preparation for Colonic Absorbed Drug

1. In blank 1.5 mL eppendorf tubes, add 300 μL of a 50/50 mixture of acetonitrile/methanol and 20 μL of the p-chlorophenylalanine as internal standard.

2. Rat blood was collected at different time points and immediately 100 μL of blood was added into the eppendorf tube and vortex to mix.

3. 10 μL of the gabapentin standard solution (0.04, 0.2, 1, 5, 25, 100 μg/mL) was added to 90 μL of blank rat blood to make up a final calibration standard (0.004, 0.02, 0.1, 0.5, 2.5, 10 μg/mL). Then 300 μL of a 50/50 mixture of acetonitrile/methanol was added into each tube followed by 20 μL of p-chlorophenylalanine.

4. Samples are vortexed and centrifuged at 14,000 rpm for 10 min.

5. Supernatant is taken for LC/MS/MS analysis.

LC/MS/MS Analysis:

API 2000 LC/MS/MS mass spectrometer equipped with Shidmadzu 10ADVp binary pumps and an autosampler (CTC Analytics AG, High Throughput Screening-PAL) were used in the analysis. A Zorbax XDB C8 4.6*150 mm column was heated to 45° C. during the analysis. The mobile phase was 0.1% formic acid (A) and acetonitrile with 0.1% formic acid (B). The gradient condition is: 5% B for 1 min, then to 98% B in 3 min and keep the same for 2.5 min. Then 5% for 2 min. A TurboIonSpray source was used on the API 2000. The analysis was done in positive ion mode and an MRM transition of 172/137 were used in the analysis of gabapentin (330/198 for Compound 1,350/198 for Compound III, 364/198 for Compound II). 20 μL of the samples were injected. The peaks were integrated by the Analyst 1.1 quantitation software.

3. Results

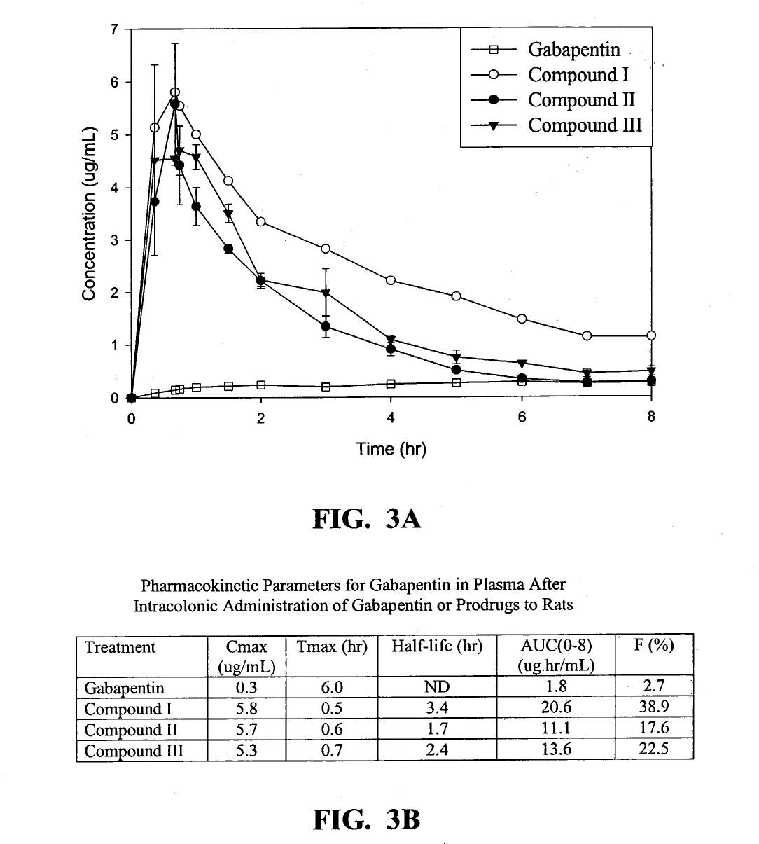

FIG. 3A compares colonic uptake of Compounds I, II and III. Uptake is determined from plasma concentration of gabapentin. It can be seen that gabapentin is not taken up significantly taken up whereas the prodrugs are taken up and converted to gabapentin with Compound I being taken up best. Uptake of the prodrugs peaks after about one hour and then gradually declines. Pharmacokinetic parameters are shown in FIG. 3B. “F” stands for oral availability. These results indicate that the conjugate moiety present in Compound I, and not present in the parent gabapentin molecule, renders the prodrug a substrate for a transporter expressed in the colon.

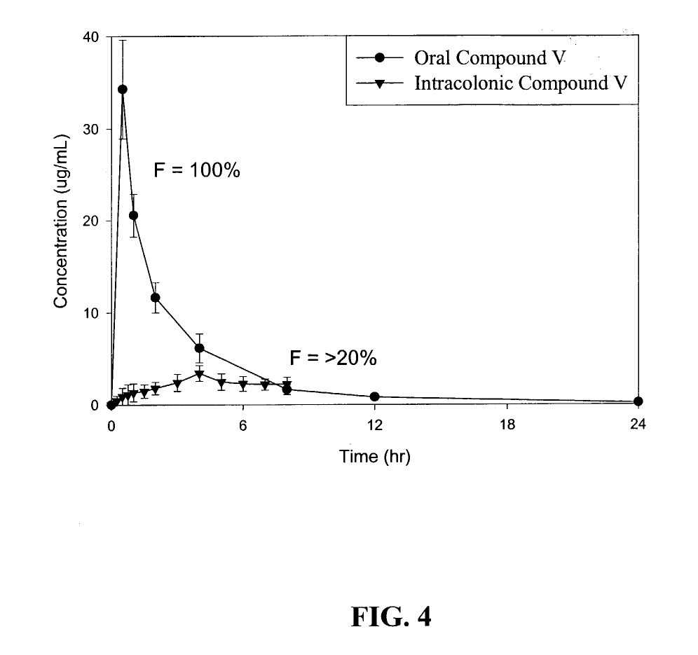

FIG. 4 compares uptake into the plasma of Compound V following oral and intracolonic administration. It can be seen that oral administration results in a rapid peak followed by a decline over the next 24 hours. Colonic dosing results in a lower peak at a later time (about 5 hr). The levels from oral and colonic administration cross at about 7 hr. This experiment indicates that uptake through the colon is useful for achieving sustained moderate levels of plasma uptake of a drug.

The above examples are illustrative only and do not define the invention; other variants will be readily apparent to those of ordinary skill in the art. The scope of the invention is encompassed by the claims of any patent(s) issuing herefrom. The scope of the invention should, therefore, be determined not with reference to the above description, but instead should be determined with reference to the issued claims along with their full scope of equivalents. Unless otherwise apparent from the context each element, feature, limitation or embodiment of the invention can be used in any combination with one another.

All publications, references, and patent documents cited in this application are incorporated by reference in their entirety for all purposes to the same extent as if each individual publication or patent document were so individually denoted.

TABLE 1

| | | | ***Expressed in humans but |
|---|---|---|---|
| | | | not other species |
| gene name | SLC Name | Genbank | *** |
| ATBO | | XM_010112 | |
| BAT | | AF141289 | *** |
| CAT-1 | SLC7A1 | XM_029358 | |
| CAT-2 | SLC7A2 | NM_003046 | *** |
| CNT1 | SLC28A1 | NM_004213 | *** |
| CNT2 | SLC28A2 | NM_004212 | *** |
| CNT3 | | NM_022127 | *** |
| FATP4 | SLC27A4 | XM_005658 | |
| GLUT-2 | SLC2A2 | NM_000340 | *** |
| GLUT-3 | SLC2A3 | NM_006931 | *** |
| GLUT-5 | SLC2A5 | NM_003039 | *** |
| MCT1 | SLC16A1 | NM-003051 | |
| MCT4 | SLC16A4 | NM-004207 | |
| NADC1 | | NM_003984 | |
| NADC2 | | NM_022444 | |
| NPT-4 | SLC17A4 | XM_030208 | *** |
| OCT_3 | SLC22A3 | NM-021977 | *** |
| OCTN1 | SLC22A4 | NP_003050 | *** |
| OCTN2 | SLC22A5 | O76082 | |
| PEPT1 | SLC15A1 | NM_005073 | |
| PGT | SLC21A2 | U70867 | |
| RBAT | SLC3A1 | L11696 | *** |
| RFC | SLC19A1 | U19720 | |
| SAT-1 | SLC26A1 | AF297659 | |
| SAT-3 | SLC26A3 | XM_004952 | |
| SAT-6 | SLC26A6 | AF416721 | |
| SERT | SLC6A4 | XM_047486 | *** |
| SGLT-1 | SLC5A1 | M24847 | *** |
| SMVT | SLC5A6 | AF081571 | |
| SUT1 | SLC13A4 | NM_012450 | *** |
| SUT2 | SLC26A2 | XM_003788 | |
| SVCT1 | SLC23A2 | AF170911 | |
| SVCT2 | SLC23A1 | AF164142 | *** |

TABLE 2

| | | | |
|---|---|---|---|
| 4F2HC | SLC3A2 | AB018010 | |
| AE1 | SLC4A1AP | XM_031667 | |
| AE2 | SLC4A2 | NM_003040 | |
| CAT-4/LAT4 | SLC7A4 | XM_036892 | |
| ENT1 | SLC29A1 | AF079117 | |
| ENT2 | SLC29A2 | NM_001532 | |
| ENT3 | SLC29A3 | AF326987 | |
| GLUT-1 | SLC2A1 | NM_006516 | *** |
| GLUT-8 | SLC2A8 | XM_011828 | |
| GLUT-13 | SLC2A13 | NM_052885 | |
| GLUT-14 | SLC2A14 | XM_016498 | |
| LAT1 | SLC7A5 | AF104032 | |
| LAT2 | SLC7A8 | NM_012244 | *** |
| MCT11 | SLC16A10 | NM_018593 | *** |
| MCT2 | SLC16A7 | NM-004731 | *** |
| MCT5 | SLC16A5 | NM-004696 | |
| MCT6 | SLC16A6 | NM-004695 | |
| MCT7 | SLC16A7 | NM-004694 | |
| NAATB | SLC1A5 | U53347 | |
| NaMI-1 | | L38500 | |
| NNaI-2 | | XP_089960 | |
| NNT-5 | | NM_014037 | |
| NNT-a | | BC006252 | *** |
| NNT-xt3 | | NM_020208 | *** |
| nSGLT-2 | | AY044906 | |
| nSGLT-3 | | AL109659 | |
| OAT-B | SLC21A9 | AB026256 | |
| OAT-D | SLC21A11 | AB031050 | |
| OAT-E | SLC21A12 | AB031051 | |
| ORCTL2 | SLC22A1L | AF037064 | |
| OST-1 | | NM_012264 | |
| OST-2 | | B1770976 | |
| OST-4 | | AI640188 | |
| PHT1 | SLC15A3 | W53019 | |
| PHT2 | | AB020598 | |
| SAT-2 | SLC26A2 | NM_000112 | |
| SGLT-2 | | AF307340 | |
| SGLT-3 | | NM_006933 | |
| SGLT-4 | SLC5A4 | SLC5A4 | *** |
| XCT | SLC7A11 | NM_014331 | *** |
| Y + LAT1 | SLC7A6 | D87432 | *** |
| Y + LAT2 | SLC7A7 | NM_003982 | *** |

1. A pharmaceutical composition comprising an agent linked to a conjugate moiety to form a conjugate, formulated with a pharmaceutical carrier for sustained or delayed release of the conjugate, wherein the conjugate has a higher Vmax for a transporter expressed in plasma membranes of epithelial cells lining a human colon than the agent alone.

2. The pharmaceutical composition of claim 1, wherein the Vmax of the conjugate is at least two-fold higher than that of the agent alone.

3. The pharmaceutical composition of claim 1, wherein the Vmax of the conjugate is at least ten-fold higher than that of the agent alone.

4. The pharmaceutical composition of claim 1, wherein the agent substantially lacks capacity to be taken up as a substrate for a transporter expressed in plasma membranes of epithelial cells lining a human colon.

5. The pharmaceutical composition of claim 1, wherein the pharmaceutical carrier comprises a polymeric material.

6. The pharmaceutical composition of claim 5, wherein the polymeric material is degraded by a change in pH, exposure to an enzyme or a change in pressure.

7. The pharmaceutical composition of claim 5, wherein the polymeric material is a non-degradable osmotic membrane.

8. The pharmaceutical composition of claim 1, wherein the agent is linked by a cleavable linkage to the conjugate moiety to form the conjugate.

9. The pharmaceutical composition of claim 1, wherein the conjugate is not a substrate for a transporter expressed in plasma membranes of epithelial cells lining a human small intestine.

10. The pharmaceutical composition of claim 1, wherein the conjugate is substantially incapable of passive transport through the human intestine.

11. The pharmaceutical composition of claim 1, wherein the conjugate has a greater Vmax for a transporter expressed in plasma membranes of epithelial cells lining a human small intestine than the agent alone.

12. The pharmaceutical composition of claim 1, wherein the agent is further linked to a second conjugate moiety to form a modified conjugate, and the modified conjugate has a reduced Vmax for a transporter expressed in plasma membranes of epithelial cells lining a human small human intestine than the conjugate alone.

13. The pharmaceutical composition of claim 1, wherein the agent is further linked to a second conjugate moiety to form a modified conjugate, and the modified conjugate has a reduced capacity for passive transport through a human intestine than the conjugate alone.

14. The pharmaceutical composition of claim 1, wherein the agent is further linked to a second conjugate moiety to form a modified conjugate, and the modified conjugate has an increased Vmax for a transporter expressed in plasma membranes of epithelial cells lining a human small human intestine than the conjugate alone.

15. The pharmaceutical composition of claim 1, wherein the transporter is selected from the group consisting of solute carrier transporters, facilitative diffusion transporters, active transporters, and pumps.

16. The pharmaceutical composition of claim 1, wherein the agent is selected from gabapentin, pregabalin and pharmaceutically acceptable salts thereof.

17. The pharmaceutical composition of claim 16, wherein the conjugate is gabapentin pivaloxymethyl carbamate, gabapentin phenylacetoxymethyl carbamate or gabapentin benzoyloxymethyl carbamate.

18. The pharmaceutical composition of claim 1, wherein the agent is selected from L-dopa, carbidopa and a pharmaceutically acceptable salts thereof.

19. The pharmaceutical composition of claim 1, wherein the transporter is a transporter described in Tables 1 or 2

20. The pharmaceutical composition of claim 1, wherein the transporter is selected from the group consisting of ATBO, CAT-1, FATP4, MCT1, MCT4, NADC1, NADC2, OCTN2, PEPT1, PGT, RFC, SAT-1, SAT-6, SMVT, SUT2 and SVCT1.

21. The pharmaceutical composition of claim 1, wherein the transporter is selected from the group consisting of MCT 1 and MCT 4.

22. The pharmaceutical composition of claim 1, wherein the transporter is selected from the group consisting of SMVT, ATBO, OCTN2, NADC1 and NADC2.

23. The pharmaceutical composition of claim 1, wherein the transporter effects transport through an apical plasma membrane or a basolateral plasma membrane of epithelial cells lining the colon, or both.

24. The pharmaceutical composition of claim 1, wherein the transporter affects transport through an apical plasma membrane of epithelial cells lining the colon.

25. A pharmaceutical composition comprising a therapeutic agent linked to a conjugate moiety to form a conjugate, formulated with a pharmaceutical carrier in an oral dosage form which upon oral administration to a human releases at least a portion of the conjugate within the colon of the human, wherein the conjugate has a higher Vmax for a transporter selected from MCT1, MCT4 and SMVT than the agent alone.

26. A method of formulating an agent, comprising:
linking the agent to a conjugate moiety to form a conjugate, wherein the conjugate moiety has a greater Vmax for a transporter expressed in plasma membranes of epithelial cells lining a human colon than the agent alone; and
formulating the conjugate with a pharmaceutical carrier as a sustained or delayed release pharmaceutical composition.

27. The method of claim 26, wherein the Vmax of the conjugate is at least two-fold higher than that of the agent alone.

28. The method of claim 26, wherein the Vmax of the conjugate is at least ten-fold higher than that of the agent alone.

29. The method of claim 26, wherein the agent substantially lacks capacity to be taken up as a substrate for a transporter expressed in plasma membranes of epithelial cells lining a human colon.

30. The method of claim 26, wherein the pharmaceutical carrier comprises a polymeric material.

31. The method of claim 30, wherein the polymeric material is degraded by a change in pH, exposure to an enzyme or a change in pressure.

32. The method of claim 30, wherein the polymeric material is a non-degradable osmotic membrane.

33. The method of claim 26, wherein the agent is linked by a cleavable linkage to the conjugate moiety to form a conjugate.

34. The method of claim 26, wherein the conjugate is not a substrate for a transporter expressed in plasma membranes of epithelial cells lining a human small intestine.

35. The method of claim 26, wherein the conjugate is substantially incapable of passive transport through the human intestine.

36. The method of claim 26, wherein the conjugate has a greater Vmax for a transporter expressed in plasma membranes of epithelial cells lining a small intestine than the agent alone.

37. The method of claim 26, wherein the agent is further linked to a second conjugate moiety to form a modified conjugate, and the modified conjugate has a reduced Vmax for a transporter expressed in plasma membranes of epithelial cells lining a small human intestine than the conjugate alone.

38. The method of claim 26, wherein the agent is further linked to a second conjugate moiety to form a modified conjugate, and the modified conjugate has a reduced capacity for passive transport through a human intestine than the conjugate alone.

39. The method of claim 26, wherein the agent is further linked to a second conjugate moiety to form a modified conjugate, and the modified conjugate has an increased Vmax for a transporter expressed in plasma membranes of epithelial cells lining a small human intestine than the conjugate alone.

40. The method of claim 26, wherein the transporter is selected from the group consisting of solute carrier transporters, facilitative diffusion transporters, active transporters, and pumps.

41. The method of claim 26, wherein the agent is selected from gabapentin, pregabalin and pharmaceutically acceptable salts thereof.

42. The method of claim 26, wherein the agent is selected from L-dopa, carbidopa and pharmaceutically acceptable salts thereof.

43. The method of claim 26, wherein the transporter is a transporter described in Tables 1 and 2.

44. The method of claim 26, wherein the transporter is selected from the group consisting of ATBO, CAT-1, FATP4, MCT1, MCT4, NADC1, NADC2, OCTN2, PEPT1, PGT, RFC, SAT-1, SAT-6, SMVT, SUT2 and SVCT1.

45. The method of claim 26, wherein the transporter is selected from the group consisting of MCT 1 and MCT 4.

46. The method of claim 26, wherein the transporter is selected from the group consisting of SMVT, ATBO, OCTN2, NADC1 and NADC2.

47. The method of claim 26, wherein the transporter effects transport through an apical plasma membrane or a basolateral plasma membrane of epithelial cells lining the colon, or both.

48. The method of claim 26, wherein the transporter effects transport through apical plasma membranes of epithelial cells lining a human colon.

49. A method of delivering an agent, comprising
orally administering to a patient a pharmaceutical composition comprising an agent linked to a conjugate moiety to form a conjugate, formulated with a pharmaceutical carrier for sustained or delayed release of the agent or conjugate, wherein the conjugate has a higher Vmax for a transporter expressed in plasma membranes of epithelial cells lining a human colon than the agent alone, whereby the conjugate is released from the carrier in the colon of the patient, and passes through the transporter into the circulation.

50. The method of claim 49, wherein the Vmax of the conjugate is at least two-fold higher than that of the agent alone.

51. The method of claim 49, wherein the Vmax of the conjugate is at least ten-fold higher than that of the agent alone.

52. The method of claim 49, wherein the agent substantially lacks capacity to be taken up as a substrate by a transporter expressed in plasma membranes of epithelial cells lining a human colon.

53. The method of claim 49, wherein the pharmaceutical carrier comprises a polymeric material.

54. The method of claim 49, wherein the polymeric material is degraded by a change in pH, exposure to an enzyme or a change in pressure.

55. The method of claim 49, wherein the polymeric material is a non-degradable osmotic membrane.

56. The method of claim 49, wherein the agent is linked by a cleavable linkage to the conjugate moiety to form the conjugate.

57. The method of claim 49, wherein the conjugate is not a substrate for a transporter expressed in plasma membranes of epithelial cells lining a human small intestine.

58. The method of claim 49, wherein the conjugate is substantially incapable of passive transport through the human intestine.

59. The method of claim 49, wherein the conjugate has a greater Vmax for a transporter expressed in plasma membranes of epithelial cells lining a human small intestine than the agent alone.

60. The method of claim 49, wherein the agent is further linked to a second conjugate moiety to form a modified conjugate, and the modified conjugate has a reduced Vmax for a transporter expressed in plasma membranes of epithelial cells lining a human small intestine than the conjugate alone.

61. The method of claim 49, wherein the agent is further linked to a second conjugate moiety to form a modified conjugate, and the modified conjugate has a reduced capacity for passive transport through a human intestine than the conjugate alone.

62. The method of claim 49, wherein the agent is further linked to a second conjugate moiety to form a modified conjugate, and the modified conjugate has an increased Vmax for a transporter expressed in plasma membranes of epithelial cells lining a human small intestine than the conjugate alone.

63. The method of claim 49, wherein the transporter is selected from the group consisting of solute carrier transporters, facilitative diffusion transporters, active transporters, and pumps.

64. The method of claim 49, wherein the agent is selected from gabapentin, pregabalin and pharmaceutically acceptable salts thereof.

65. The method of claim 49, wherein the conjugate is gabapentin pivaloxymethyl carbamate, gabapentin phenylacetoxymethyl carbamate or gabapentin benzoyloxymethyl carbamate.

66. The method of claim 49, wherein the agent is selected from L-dopa, carbidopa and pharmaceutically acceptable salts thereof.

67. The method of claim 49, wherein the transporter is a transporter described in Table 1.

68. The method of claim 49, wherein the transporter is selected from the group consisting of ATBO, CAT-1, FATP4, MCT1, MCT4, NADC1, NADC2, OCTN2, PEPT1, PGT, RFC, SAT-1, SAT-6, SMVT, SUT2 and SVCT1.

69. The method of claim 49, wherein the transporter is selected from the group consisting of MCT 1 and MCT 4.

70. The method of claim 49, wherein the transporter is selected from the group consisting of SMVT, ATBO, OCTN2, NADC1 and NADC2.

71. A method of screening agents, conjugates or conjugate moieties for oral delivery, comprising
providing a cell expressing a transporter expressed in the human colon, the transporter being situated in the plasma membrane of the cell;
contacting the cell with an agent, conjugate or conjugate moiety; and
determining whether the agent, conjugate or conjugate moiety passes through the plasma membrane via the transporter.

72. The method of claim 71, wherein the agent or conjugate is substantially incapable of passive diffusion through the plasma membrane.

73. A method of delivering an agent, comprising
orally administering to a patient a pharmaceutical composition comprising an agent, optionally, linked to a conjugate moiety to form a conjugate, formulated with a pharmaceutical carrier for sustained or delayed release of the agent or conjugate, wherein the agent, conjugate moiety (if present) or conjugate (if present) has been screened to determine that it is a substrate for a transporter expressed in plasma membranes of epithelial cells lining a human colon.

74. The method of claim 73, wherein the screening was performed by
providing a cell expressing a transporter expressed in plasma membranes of epithelial cells lining a human colon, the transporter being situated in the plasma membrane of the provided cell;
contacting the provided cell with an agent, conjugate or conjugate moiety; and
determining whether the agent, conjugate or conjugate moiety passes through the membrane via the transporter.

75. The method of claim 73, wherein the pharmaceutical carrier comprises a polymeric material.

76. The method of claim 73, wherein the polymeric material is degraded by a change in pH, exposure to an enzyme or a change in pressure.

77. The pharmaceutical composition of claim 73, wherein the polymeric material is a non-degradable osmotic membrane.

78. The method of claim 73, wherein the agent or conjugate (if present) is not a substrate for a transporter expressed in plasma membranes of epithelial cells lining a human small intestine.

79. The method of claim 73, wherein the agent or conjugate (if present) is substantially incapable of passive transport through the human intestine.

80. The method of claim 73, wherein the transporter is selected from the group consisting of solute carrier transporters, facilitative diffusion transporters, active transporters, and pumps.

81. The method of claim 73, wherein the transporter is a transporter described in Table 1.

82. The method of claim 73, wherein the transporter is selected from the group consisting of ATBO, CAT-1, FATP4, MCT1, MCT4, NADC1, NADC2, OCTN2, PEPT1, PGT, RFC, SAT-1, SAT-6, SMVT, SUT2 and SVCT1.

83. The method of claim 73, wherein the transporter is selected from the group consisting of MCT 1 and MCT 4.

84. The method of claim 73, wherein the transporter is selected from the group consisting of SMVT, ATBO, OCTN2, NADC1 and NADC2.

85. The method of claim 73, wherein the transporter effects transport through an apical plasma membrane or a basolateral plasma membrane of epithelia cells lining the colon, or both.

86. The method of claim 73, wherein the transporter effects transport through apical plasma membranes of epithelial cells lining the colon.
